(19) United States
(12) Patent Application Publication
(10) Pub. No.: US 2008/0095743 A1
(43) Pub. Date: Apr. 24, 2008

(54) Monoclonal Antibody that Specifically Binds Stem Cells and its Use

(75) Inventors: Neal D. Epstein, Chevy Chase, MD (US); Steve O. Winitsky, Bethesda, MD (US); Thiru V. Gopal, North Potomac, MD (US); Shahin Hassanzadeh, Fairfax, VA (US)

Correspondence Address: KLARQUIST SPARKMAN, LLP, 121 S.W. SALMON STREET, SUITE #1600, PORTLAND, OR, 97204-2988, US

(73) Assignee: THE GOVERNMENT OF THE UNITED STATES OF AMERICA AS, Rockville, MD (US)

(21) Appl. No.: 11/578,891

(22) PCT Filed: Apr. 25, 2005
(86) PCT No.: PCT/US05/14176

§ 371 Date: Sep. 7, 2007

Related U.S. Application Data
(60) Provisional application No. 60/565,101, filed on Apr. 23, 2004.

(51) Int. Cl. A61K 48/00; A61P 25/00; C12N 5/02; C12N 5/04; G01N 33/53

(52) U.S. Cl. 424/93.2; 435/368; 435/378; 435/383; 435/395; 435/7.21

(57) ABSTRACT

Antibodies are disclosed herein that bind Spoc cells. In one embodiment the antibodies are monoclonal antibodies. The use of antibodies that bind Spoc cells to identify and/or isolate a sub-population of Spoc cells is also disclosed. In one embodiment, a method for treating a neurologic disorder is provided. The method includes administering a sub-population of Spoc cells and/or and neuronal cells differentiated from Spoc cells to treat a neurologic disorder.

### PRIORITY CLAIM

This application claims the benefit of U.S. Provisional Application No. 60/565,101, filed Apr. 23, 2004, which is incorporated herein by reference.

### FIELD

This disclosure relates to the field of neuronal precursor cells, specifically to the use of an antibody that specifically binds Spoc cells to identify and isolate neuronal precursor cells.

### BACKGROUND

Neurons in the central and peripheral nervous systems degenerate as a normal function of human development and aging. Pathological neuron degeneration, however, is a serious condition seen in several neurological disorders. Neuronal degeneration can be specific or diffuse, and can lead to sensory, motor and cognitive impairments. Neurodegenerative disorders encompass a range of seriously debilitating conditions including Parkinson's disease, amyotrophic lateral sclerosis (ALS, “Lou Gehrig's disease”), multiple sclerosis, Huntington's disease, Alzheimer's disease, Pantothenate kinase associated neurodegeneration (PKAN, formerly Hallervorden-Spatz syndrome), multiple system atrophy, diabetic retinopathy, multi-infarct dementia, macular degeneration, and the like. These conditions are characterized by a gradual but relentless worsening of the patient's condition over time. These disorders affect a large population of humans, especially older adults. Nevertheless, the understanding of these disorders is extremely limited and incomplete.

Many advances have been made in years past in gaining a better understanding of Parkinson's disease, Alzheimer's disease, and Huntington's disease. The primary cause of cognitive dysfunction for all three disorders has been directly linked to neuron degeneration, usually in specific areas of the brain. Parkinson's disease is linked to degeneration of neurons in the substantia nigra, while Alzheimer's disease is in some part due to loss of pyramidal neurons in the limbic cortex (Braak, E. & Braak, H., in: V. E. Koliatsos & R. R. Ratan (eds.), Cell Death and Diseases of the Nervous System, Totowa, N. J.: Humana Press, pp. 497-508, 1999). Huntington's disease's cognitive deficits are produced by degeneration of cells in the caudate nucleus of the striatum. However, although the symptoms and progression of these diseases are well characterized, the causes and triggers at onset are not well understood.

Thus, several strategies are being pursued to develop new therapies for neurodegenerative disorders, including Parkinson's disease. For Parkinson's disease, the techniques range from the use of dopaminotrophic factors (Takayama et al., Nature Med. 1:53-58, 1995) and viral vectors (Choi-Lundberg et al., Science 275:838-841, 1997) to the transplantation of primary xenogenic tissue (Deacon et al., Nature Med. 3:350-353, 1997). Transplantation of dopaminergic neurons is a clinically promising experimental treatment in late stage Parkinson's disease. More than 200 patients have been transplanted worldwide (Olanow et al., Trends Neurosci. 19:102-109, 1996), and clinical improvement has been confirmed (Olanow et al, supra, and Wenning et al., Ann. Neurol. 42:95-107, 1997) and was correlated to good graft survival and innervation of the host striatum (Kordower et al., N. Engl. J. Med. 332:1118-1124, 1995). However, fetal nigral transplantation therapy generally requires human fetal tissue from at least 3-5 embryos to obtain a clinically reliable improvement in the patient. A different source of neurons is clearly needed.

Embryonic stem (ES) cells, derived from the inner cell mass of the blastocyst, are the most primitive stem cell. These cells have unlimited self-renewal capability, and because they can differentiate into several cell lineages and repopulate tissues upon transplantation, they have multipotent differentiative potential. It has been proposed that ES cells can be used to produce differentiated cell types, such as neurons.

Lineage specific stem cells, identified in most organ tissues, have less self-renewal capability than ES cells and their differentiative ability is limited to tissues of that lineage. Of the lineage specific stem cells, the hematopoietic stem cell (HSC), derived from bone marrow, blood, cord blood, fetal liver and yolk sack, is the best characterized. These cells are defined by the expression of cell surface markers, such as c-kit (c-kit⁺), and can terminally differentiate into all the hematopoietic cell types. HSC have been shown to contribute to the formation of functional cardiac tissue in vivo (Jackson et al., J. Clin. Invest., 107:1395-1402, 2001). Mesenchymal stem cells (MSC) are pluripotent progenitor cells derived from tissues of mesodermal origin (U.S. Pat. No. 5,486,359). These cells are most often obtained from bone marrow, although they can be obtained from other sources, such as blood or dermis. These cells have been shown to differentiate to form muscle, bone, cartilage, fat, marrow stroma and tendon, but have not been shown to differentiate into cardiomyocytes or neurons. Thus, there is a need to identify a novel source of stem cells that can differentiate into cardiomyocytes or neurons.

### SUMMARY

Neuronal precursor cells differentiate into neurons. These cells are of use for screening agents that affect the nervous system. As disclosed herein, antibodies can be used to identify these cells from a mixed population of cells and to isolate these cells for in vitro culture systems and for in vivo use.

Skeletal-based precursor of cardiomyocytes (Spoc) cells are derived from skeletal muscle, are from about 3 μm to about 10 μm in diameter, and do not express the cell surface markers c-met, c-kit, CD34, or Sca-1, or the Pax 3 and Pax 7 transcription factors (Pax(3/7)). It is disclosed herein that Spoc cells can differentiate in vitro to form a fully functional cell of more than one given cell type. Specifically, it is demonstrated herein that Spoc cells can be differentiated into either cardiomyocytes or neurons.

Antibodies are disclosed herein that bind c-kit⁻c-met⁻CD34⁻Sca-1⁻Pax(3/7)⁻Spoc cells. These antibodies can be used to identify and isolate a sub-population of Spoc cells or neuronal precursor cells that differentiate into neurons. The use of these antibodies allows the production of cultures that can be used to screen agents to identify those agents that affect neuronal cell function or differentiation. Thus, use of these antibodies allows the identification of cells derived from Spoc cells that can be used to treat neurologic disorders. For example, a therapeutically effective amount of a Spoc cell subpopulation can be administered to a subject with a neurologic disorder to reduce a sign or symptom of the disorder.

The foregoing and other features and advantages will become more apparent from the following detailed description of several embodiments, which proceeds with reference to the accompanying figures.

### BRIEF DESCRIPTION OF THE FIGURES

FIG. 1 is series of digital images of murine Spoc cells immunostained with monoclonal antibody 804 at different days in culture. The murine Spoc cells are stained at day 0 (FIG. 1A), day 2 (FIG. 1B), and day 4 (FIG. 1C). Examples of cells that express the antigen recognized by the 804 monoclonal antibody are indicated by arrows in these images.

FIG. 2 is a series of digital images of cells immunostained with the 804 monoclonal antibody. FIG. 2A is a section of murine skeletal muscle. FIG. 2B is a digital image of a section of human biceps (skeletal muscle). The cells in FIGS. 2A and 2B were immunostained with the 804 monoclonal antibody and a second fluorescently labeled antibody that specifically binds the 804 monoclonal antibody. Examples of cells that express the antigen recognized by the 804 monoclonal antibody are indicated by arrows in these images.

FIG. 3 is a series of digital images of immunostained cells. FIG. 3A is a digital image of a 12 day culture of pig 804+ cells from skeletal muscle growing as a neurosphere and showing neuron-specific expression (beta-3-tubulin) at the peripherae. Cells that are stained for beta-3-tubulin are indicated by arrows. FIG. 3B is a digital image of a section of murine spinal cord immunostained with monoclonal antibody 804 and a second fluorescent labeled antibody that specifically binds monoclonal antibody 804. Examples of cells that express the antigen recognized by the 804 monoclonal antibody are indicated by arrows in this image.

FIG. 4 is a series of digital images of immunostained cells. FIG. 4A is a digital image of dissociated mouse embryo cells, isolated with the 804 monoclonal antibody, after 14 days in culture and stained with the 804 monoclonal antibody. FIG. 4B is a digital image of 804⁺ cells isolated from mouse skeletal muscle and co-stained with the 804 antibody and the GRIN1 antibody. Examples of cells that are bound by both antibodies are indicated by arrows.

FIG. 5 is a series of digital images demonstrating the immunoprecipitation and expression of the antigen recognized by the 804 monoclonal antibody. FIG. 5A shows that the GRIN1 antibody immunoprecipitates a protein that can be detected by the 804 monoclonal antibody and the GRIN1 antibody. FIG. 5B is a digital image demonstrating that, by RT-PCR, GRIN1 is expressed in Spoc cells and in the hippocampus.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NOs: 1-2 are amino acid sequences of murine GRIN1 polypeptides.

SEQ ID NOs: 3-10 are amino acid sequences of subunit isoforms of the rat NMDAR1 polypeptide.

SEQ ID NO: 11 is an amino acid sequence of a human GRIN1 polypeptide.

SEQ ID NOs: 12-14 are amino acid sequences of human GRIN1 isoforms.

SEQ ID NOs: 15-18 are amino acid sequences of murine GRIN1 peptides.

### DETAILED DESCRIPTION

I. Abbreviations

CS: Cardiac precursors from Spoc cells

DNA: Deoxyribonucleic acid

EGF: Epidermal growth factor

EGFP: Enhanced green fluorescent protein

ES: Embryonic stem

FACS: Fluorescence activated cell sort

FBS: Fetal bovine serum

FGF: Fibroblast growth factor

HSC: Hematopoietic stem cell

RNA: Messenger ribonucleic acid

PBS: Phosphate buffered saline

RNase: Ribonuclease

RT-PCR: Reverse transcriptase-polymerase chain reaction

SPOC: Skeletal-based precursors of cardiomyocytes

II. Terms

Unless otherwise noted, technical terms are used according to conventional usage. Definitions of common terms in molecular biology may be found in Benjamin Lewin, Genes V, published by Oxford University Press (ISBN 0-19-854287-9), 1994; Kendrew et al. (eds.), The Encyclopedia of Molecular Biology, published by Blackwell Science Ltd. (ISBN 0-632-02182-9), 1994; and Robert A. Meyers (ed.), Molecular Biology and Biotechnology: a Comprehensive Desk Reference, published by VCH Publishers, Inc. (ISBN 1-56081-569-8), 1995.

In order to facilitate review of the various embodiments of this disclosure, the following explanations of specific terms are provided:

Adult: A fully developed and physically mature subject, having attained full size and strength.

Animal: Living multi-cellular vertebrate organisms, a category that includes, for example, mammals and birds.

Antibody: Immunoglobulin molecules and immunologically active portions of immunoglobulin molecules, for instance, molecules that contain an antigen binding site that specifically binds (immunoreacts with) an antigen.

A naturally occurring antibody (for example, IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody.

Immunoglobulins and certain variants thereof are known, and many have been prepared in recombinant cell culture (for example, see U.S. Pat. No. 4,745,055; U.S. Pat. No. 4,444,487; WO 88/03565; EP 256,654; EP 120,694; EP 125, 023; Faoulkner et al., Nature 298:286, 1982; Morrison, J. Immunol. 123:793, 1979; Morrison et al., Ann Rev. Immunol. 2:239, 1984).

Antibody fragment (fragment with specific antigen binding): Various fragments of antibodies have been defined, including Fab, (Fab′)₂, Fv, and single-chain Fv (scFv). These antibody fragments are defined as follows: (1) Fab, the fragment that contains a monovalent antigen-binding fragment of an antibody molecule produced by digestion of whole antibody with the enzyme papain to yield an intact light chain and a portion of one heavy chain or equivalently by genetic engineering; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction or equivalently by genetic engineering; (4) F(Ab′)₂, a dimer of two FAb′ fragments held together by disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. Methods of making these fragments are routine in the art.

Antigen: A molecule (for example, polypeptide) that is specifically recognized and bound by an antibody. Those antigens that can induce antibody production are called immunogens.

Avidity: The overall strength of interaction between two molecules, such as an antigen and an antibody. Avidity depends on both the affinity and the valency of interactions. Therefore, the avidity of a pentameric IgM antibody, with ten antigen binding sites, for a multivalent antigen may be much greater than the avidity of a dimeric IgG molecule for the same antigen.

Cardiac muscle: The heart is made of specialized muscle tissue with some similarities to both smooth and skeletal muscle. It is involuntary and mononucleate, as is smooth muscle. Cardiac muscle is striated like skeletal muscle, which means that it has microscopically visible myofilaments arranged in parallel with the sarcomere. These filaments slide along each other during the process of contraction in the same manner as occurs in skeletal muscle. However, cardiac muscle contains more mitochondria so the striations are not as organized as they are in skeletal muscle. Cardiac muscle also differs from skeletal muscle in that the fibers in cardiac muscle branch and usually have a single centrally located nucleus. Another difference in cardiac muscle is the presence of intercalated discs which serve as specialized connections between cardiac muscle cells. These tight connections allow for almost completely free movement of ions so that action potentials can freely pass from one cell to another. This arrangement makes cardiac muscle tissue a functional syncytium. When one cell is excited, the resultant action potential is spread to all of them. This is an important feature in that it allows the atrial or ventricular muscle to contract as a unit to forcefully pump blood. Cardiac muscle can generate its own excititory impulses from the sin θ-atrial node, which acts like a biological pacemaker. In this manner, the contracting signal for cardiac muscles originates in the heart itself. However, the autonomic nervous system (for example through the vagus nerve) can exert control over how fast the signals form and propagate through the heart, which regulates the rate of myocardial contraction. A “cardiomyocyte” is a cell of the cardiac muscle.

Cardiac precursors from Spoc cells (CS cells): When Spoc cells are isolated from skeletal muscle and are cultured under growth conditions designed to promote their growth, Spoc cells undergo several rounds of division. During this proliferative phase they become clusters of floating round cells with an increased diameter as compared to Spoc cells. These round cells, with an increased diameter, are referred to as CS cells. In one embodiment, a diameter of a CS cell is from about 10 to about 14 μm. When placed in growth promoting conditions in vitro CS cells differentiate into spontaneously beating cardiomyocytes. A subset of CS cells can differentiate into cells with a neuronal phenotype.

Cell surface marker: A protein, glycoprotein, or other molecule expressed on the surface of a cell, which serves to help identify the cell. A cell surface marker can generally be detected by conventional methods. Specific, non-limiting examples of methods for detection of a cell surface marker are immunohistochemistry, fluorescence activated cell sorting (FACS), or an enzymatic analysis.

Central Nervous System (CNS): The part of the nervous system of an animal that contains a high concentration of cell bodies and synapses and is the main site of integration of nervous activity. In higher animals, the CNS generally refers to the brain and spinal cord.

Complementarity-determining region (CDR): The CDRs are three hypervariable regions within each of the variable light (V_{L}) and variable heavy (V_{H}) regions of an antibody molecule that form the antigen-binding surface that is complementary to the three-dimensional structure of the bound antigen. Proceeding from the N-terminus of a heavy or light chain, these complementarity-determining regions are denoted as “CDR1”, “CDR2,” and “CDR3,” respectively. CDRs are involved in antigen-antibody binding, and the CDR3 comprises a unique region specific for antigen-antibody binding. An antigen-binding site, therefore, may include six CDRs, comprising the CDR regions from each of a heavy and a light chain V region. Alteration of a single amino acid within a CDR region can destroy the affinity of an antibody for a specific antigen (see Abbas et al., Cellular and Molecular Immunology, 4th ed. 143-5, 2000). The locations of the CDRs have been precisely defined, for example, by Kabat et al., Sequences of Proteins of Immunologic Interest, U.S. Department of Health and Human Services, 1983.

Differentiation: The process whereby relatively unspecialized cells (for example, stem cells) acquire specialized structural and/or functional features characteristic of mature cells. Similarly, “differentiate” refers to this process. Typically, during differentiation, cellular structure alters and tissue-specific proteins appear. The term “differentiated muscle cell” refers to cells expressing a protein characteristic of the specific muscle cell type. A differentiated muscle cell includes a skeletal muscle cell, a smooth muscle cell, and a cardiac muscle cell.

Differentiation medium: A synthetic set of culture conditions with the nutrients necessary to support the growth or survival of cultured cells, and which allows the differentiation of stem cells into differentiated cells.

DNA: Deoxyribonucleic acid. DNA is a long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide. The term codon is also used for the corresponding (and complementary) sequences of three nucleotides in the mRNA into which the DNA sequence is transcribed.

Epidermal growth factor (EGF): In particular examples, EGF is a globular protein of 6.4 kDa consisting of 53 amino acids. It contains three intramolecular disulfide bonds essential for biological activity. EGF proteins are evolutionarily closely conserved. Human EGF and murine EGF have 37 amino acids in common. Approximately 70 percent homology is found between human EGF and EGF isolated from other species. Mammalian EGF includes, but is not limited to, murine, avian, canine, bovine, porcine, equine, and human EGF. The amino acid sequences and methods for making these EGF polypeptides are well known in the art.

The gene encoding the EGF precursor has a length of approximately 110 kb, and contains 24 exons. Fifteen of these exons encode protein domains that are homologous to domains found in other proteins. The human EGF gene maps to chromosome 4q25-q27.

EGF is a strong mitogen for many cells of ectodermal, mesodermal, and endodermal origin. EGF controls and stimulates the proliferation of epidermal and epithelial cells, including fibroblasts, kidney epithelial cells, human glial cells, ovary granulosa cells, and thyroid cells in vitro. EGF also stimulates the proliferation of embryonic cells. However, the proliferation of some cell lines has been shown to be inhibited by EGF.

EGF is also known to act as a differentiation factor for some cell types. It strongly influences the synthesis and turn-over of proteins of the extra-cellular matrix including fibronectin, collagen, laminin, and glycosaminoglycans, and has been shown to be a strong chemoattractant for fibroblasts and epithelial cells.

EGF can be assayed in a cell-based assay wherein the proliferation of a cell population is assessed. EGF can also be assayed by an immunoassay, such as an ELISA assay.

Fragments of EGF, smaller than the full-length sequence can also be employed in methods disclosed herein. Suitable biologically active variants can also be utilized. One specific, non-limiting example of an EGF variant of use is an EGF sequence having one or more amino acid substitutions, insertions, or deletions, wherein a biological function of EGF is retained. Another specific, non-limiting example of an EGF variant is EGF wherein glycosylation or phosphorylation is altered, or a foreign moiety is added, so long as a biological function of EGF is retained. Methods for making EGF fragments, analogs, and derivatives are available in the art. Examples of EGF variants are known in the art, for example U.S. Pat. No. 5,218,093 and WO 92/16626A1. Examples of EGF from many different species are disclosed in WO 92/16626A1, as are examples of variants, and strategies for producing them.

As used herein, “EGF” refers to naturally occurring EGF, and variants and fragments that perform the same function of EGF in the culture media disclosed herein.

Embryonic stem (ES) cells: Totipotent cells isolated from the inner cell mass of the developing blastocyst and can generate all of the cells present in the body (bone, muscle, brain cells, etc.). “ES cells” can be derived from any organism, for example from mammals such as humans.

Epitope: The site on an antigen recognized by an antibody as determined by the specificity of the amino acid sequence. Two antibodies are said to bind to the same epitope if each competitively inhibits (blocks) binding of the other to the antigen as measured in a competitive binding assay (see, for example, Junghans et al., Cancer Res. 50:1495-1502, 1990). Alternatively, two antibodies have the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

Expand: A process by which the number or amount of cells in a cell culture is increased due to cell division. Similarly, the terms “expansion” or “expanded” refers to this process. The terms “proliferate,” “proliferation” or “proliferated” may be used interchangeably with the words “expand,” “expansion” or “expanded.”

Fibroblast growth factor (FGF): Any suitable fibroblast growth factor, derived from any animal, and functional variants and fragments thereof. A variety of FGFs are known and include, but are not limited to, FGF-1 (acidic fibroblast growth factor), FGF-2 (basic fibroblast growth factor, bFGF), FGF-3 (int-2), FGF-4 (hst/K-FGF), FGF-5, FGF-6, FGF-7, FGF-8, and FGF-9. FGF refers to a fibroblast growth factor protein such as FGF-1, FGF-2, FGF-4, FGF-6, FGF-8, or FGF-9, or a biologically active fragment or mutant thereof. The FGF can be from any animal species. In one embodiment the FGF is mammalian FGF, including but not limited to, rodent, avian, canine, bovine, porcine, equine, and human. The amino acid sequences and method for making many of the FGFs are well known in the art.

Fragments of FGF that are smaller than those described can also be employed.

Suitable biologically active variants can be FGF analogs or derivatives. An analog of FGF is either FGF or an FGF fragment that includes a native FGF sequence and structure having one or more amino acid substitutions, insertions, or deletions. Analogs having one or more peptoid sequences (peptide mimic sequences) are also included (see for example, PCT Publication No. WO 91/04282). By “derivative” is intended any suitable modification of FGF, FGF fragments, or their respective analogs, such as glycosylation, phosphorylation, or other addition of foreign moieties, as long as the FGF activity is retained. Methods for making FGF fragments, analogs, and derivatives are available in the art.

Framework region (FR): Relatively conserved sequences flanking the three highly divergent complementarity-determining regions (CDRs) within the variable regions of the heavy and light chains of an antibody. Hence, the variable region of an antibody heavy or light chain consists of a FR and three CDRs. Some FR residues may contact bound antigen; however, FRs are primarily responsible for folding the variable region into the antigen-binding site, particularly the FR residues directly adjacent to the CDRs. Without being bound by theory, the framework region of an antibody serves to position and align the CDRs. The sequences of the framework regions of different light or heavy chains are relatively conserved within a species. A “human” framework region is a framework region that is substantially identical (about 85% or more, usually 90-95% or more) to the framework region of a naturally occurring human immunoglobulin.

Glutamate Receptor, Ionotropic, N-methyl D-aspartate 1 (GRIN1): A protein that is a critical subunit of N-methyl-D-aspartate receptors. GRIN1 can also be referred to as N-methyl-D-aspartate receptor channel, subunit zeta-1, NMDAR1, or NR1. Members of the glutamate receptor channel superfamily are heteromeric protein complexes with multiple subunits arranged to form a ligand-gated ion channel. These subunits are believed to play a key role in the plasticity of synapses, which is believed to underlie memory and learning.

The gene encoding GRIN1 consists of 21 exons and is alternatively spliced, producing transcript variants differing in the C-terminus. The human GRIN1 splice variant NR1-1 is missing exon 20 and is alternately spliced in exon 21, leading to the short isoform NR1-1 which has a different C-terminus. The human GRIN1 splice variant NR1-2 is missing exon 20 and is spliced in exon 21, leading to the medium isoform NR1-2. The human GRIN1 splice variant NR1-3 includes all 21 exons with alternate splicing of exon 21 matching that of transcript variant NR1-2, resulting in the long isoform. Although the sequence of exon 5 is identical in human and rat, the alternative exon 5 splicing in rat has yet to be demonstrated in humans. In the rat, eight splice variants have been identified (NMDAR1-1a, 1b, 2a, 2b, 3a, 3b, 4a, and 4b).

Growth factor: A substance that promotes cell growth, survival, and/or differentiation. In general, growth factors stimulate cell proliferation or maturation when they bind to their receptor. In one embodiment, growth factors are a complex family of polypeptide hormones or biological factors that control growth, division, and maturation of muscle cells. In another embodiment a growth factor can be used to promote the proliferation of muscle stem cells and maintain the stem cells in an undifferentiated state. A growth factor can be a naturally occurring factor or a factor synthesized using molecular biology techniques. Examples of growth factors include platelet-derived growth factor, fibroblast growth factor, epidermal growth factor, insulin, somatomedin, stem cell factor, vascular endothelial growth factor, granulocyte colony stimulating factor, and transforming growth factor-beta, amongst others. A muscle cell growth factor is a growth factor that effects the development (maturation), differentiation, division, or proliferation of muscle cells.

Growth medium: A synthetic set of culture conditions with the nutrients necessary to support the growth or survival of microorganisms or cultured cells.

Heart: The muscular organ of an animal that circulates blood. The walls of the heart are comprised of working muscle, or myocardium, and connective tissue. Myocardium is comprised of myocardial cells, which are also referred to herein as cardiac cells, cardiac myocytes, cardiomyocytes and/or cardiac fibers. Cardiomyocytes may be cells of the atrium or cells of the ventricle. “Cardiac cells” refers to the cells of the heart.

Heterologous: A heterologous sequence is a sequence that is not normally (for instance, in the wild-type sequence) found adjacent to a second sequence. In one embodiment, the sequence is from a different genetic source, such as a virus or organism, than the second sequence.

Immunoglobulin: A protein including one or more polypeptides substantially encoded by immunoglobulin genes. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 K_{d} or 214 amino acids in length. Full-length immunoglobulin heavy chains are generally about 50 K_{d} or 446 amino acid in length. Light chains are encoded by a variable region gene at the NH2-terminus (about 110 amino acids in length) and a kappa or lambda constant region gene at the COOH—tenninus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (for example, Lanzavecchia et al., Eur. J. Immunol. 17:105, 1987; Huston et al., Proc. Natl. Acad. Sci. U.S.A. 85:5879-5883, 1988; Bird et al., Science 242:423-426, 1988; Hood et al., Immunology, Benjamin, N.Y., 2nd ed., 1984; Hunkapiller and Hood, Nature 323:15-16, 1986).

An immunoglobulin light or heavy chain variable region includes a framework region interrupted by three hypervariable regions, also called complementarity determining regions (CDRs) (see, Sequences of Proteins of Immunological Interest, E. Kabat et al., U.S. Department of Health and Human Services, 1983). As noted above, the CDRs are primarily responsible for binding to an epitope of an antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody (for example, ATCC Accession No. CRL 9688 secretes an anti-Tac chimeric antibody), although other mammalian species can be used, or the variable region can be produced by molecular techniques. Methods of making chimeric antibodies are well known in the art, for example, see U.S. Pat. No. 5,807,715, which is herein incorporated by reference.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, for instance, at least about 85-90%, such as about 95% or more identical. The donor CDRs of a humanized antibody can have a limited number of substitutions using amino acids from the acceptor CDR. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody can have a limited number of substitutions by amino acids taken from the donor framework. Humanized or other monoclonal antibodies can have additional amino acid substitutions which have substantially no effect on antigen binding or other immunoglobulin functions. Exemplary conservative substitutions are those such as gly, ala; val, ile, leu; asp, glu; asn, gln; ser, thr; lys, arg; and phe, tyr (see U.S. Pat. No. 5,585,089, which is incorporated herein by reference). Humanized immunoglobulins can be constructed by means of genetic engineering, for example, see U.S. Pat. No. 5,225,539 and U.S. Pat. No. 5,585,089, which are herein incorporated by reference.

A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, for example, Dower et al., PCT Publication No. WO91/17271; McCafferty et al., PCT Publication No. WO92/001047; and Winter, PCT Publication No. WO92/20791, which are herein incorporated by reference), or selected from a human combinatorial monoclonal antibody library (see the Morphosys website). Human antibodies can also be prepared by using transgenic animals carrying a human immunoglobulin gene (for example, see Lonberg et al., PCT Publication No. WO93/12227; and Kucherlapati, PCT Publication No. WO91/10741, which are herein incorporated by reference).

Isolated: An “isolated” biological component (such as a nucleic acid molecule, protein or organelle) has been substantially separated or purified away from other biological components in the cell of the organism in which the component naturally occurs, for instance, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Mammal: This term includes both human and non-human mammals. Similarly, the term “subject” includes both human and veterinary subjects.

Monoclonal antibody: An antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells.

Muscle cell: Includes skeletal, cardiac or smooth muscle tissue cells. This term is synonymous with myocyte, and encompasses those cells which differentiate to form more specialized muscle cells (for example, myoblasts). “Cardiomyocyte” refers to a cardiac muscle cell.

Neurological disorder: A disorder in the nervous system, including the central nervous system (CNS) and peripheral nervous system (PNS). Examples of neurological disorders include Parkinson's disease, Huntington's disease, Alzheimer's disease, severe seizure disorders including epilepsy, familial dysautonomia as well as injury or trauma to the nervous system, such as neurotoxic injury or disorders of mood and behavior such as addiction, schizophrenia and amyotrophic lateral sclerosis. Neuronal disorders also include Lewy body dementia, multiple sclerosis, epilepsy, cerebellar ataxia, progressive supranuclear palsy, amyotrophic lateral sclerosis, affective disorders, anxiety disorders, obsessive compulsive disorders, personality disorders, attention deficit disorder, attention deficit hyperactivity disorder, Tourette Syndrome, Tay Sachs, Nieman Pick, and other lipid storage and genetic brain diseases and/or schizophrenia. A “neurodegenerative disorder” is an abnormality in the nervous system of a subject, such as a mammal, in which neuronal integrity is threatened. Without being bound by theory, neuronal integrity can be threatened when neuronal cells display decreased survival or when the neurons can no longer propagate a signal. Specific, non-limiting examples of a neurodegenerative disorder are Alzheimer's disease, Pantothenate kinase associated neurodegeneration, Parkinson's disease, Huntington's disease (Dexter et al., Brain 114:1953-1975, 1991), HIV encephalopathy (Miszkziel et al., Magnetic Res. Imag. 15:1113-1119, 1997), and amyotrophic lateral sclerosis.

Alzheimer's disease manifests itself as pre-senile dementia. The disease is characterized by confusion, memory failure, disorientation, restlessness, speech disturbances, and hallucination in mammals (Medical, Nursing, and Allied Health Dictionary, 4th Ed., 1994, Editors: Anderson, Anderson, Glanze, St. Louis, Mosby).

Parkinson's disease is a slowly progressive, degenerative, neurologic disorder characterized by resting tremor, loss of postural reflexes, and muscle rigidity and weakness (Medical, Nursing, and Allied Health Dictionary, 4th Ed., 1994, Editors: Anderson, Anderson, Glanze, St. Louis, Mosby).

Amyotrophic lateral sclerosis is a degenerative disease of the motor neurons characterized by weakness and atrophy of the muscles of the hands, forearms, and legs, spreading to involve most of the body and face (Medical, Nursing, and Allied Health Dictionary, 4th Ed., 1994, Editors: Anderson, Anderson, Glanze, St. Louis, Mosby).

Pantothenate kinase associated neurodegeneration (PKAN, also known as Hallervorden-Spatz syndrome) is an autosomal recessive neurodegenerative disorder associated with brain iron accumulation. Clinical features include extrapyramidal dysfunction, onset in childhood, and a relentlessly progressive course (Dooling et al., Arch. Neurol. 30:70-83, 1974). PKAN is a clinically heterogeneous group of disorders that includes classical disease with onset in the first two decades, dystonia, high globus pallidus iron with a characteristic radiographic appearance (Angelini et al., J. Neurol. 239:417-425, 1992), and often either pigmentary retinopathy or optic atrophy (Dooling et al., Arch. Neurol. 30:70-83, 1974; Swaiman et al., Arch. Neurol 48:1285-1293, 1991).

A “neurodegenerative related disorder” is a disorder such as speech disorders that are associated with a neurodegenerative disorder. Specific non-limiting examples of a neurodegenerative related disorders include, but are not limited to, palilalia, tachylalia, echolalia, gait disturbance, perseverative movements, bradykinesia, spasticity, rigidity, retinopathy, optic atrophy, dysarthria, and dementia.

N-methyl-D-aspartate (NMDA) Receptor: An ionotropic receptor for glutamate which is a member of the glutamate receptor channel superfamily. The members of the superfamily are heteromeric protein complexes with multiple subunits arranged to form a ligand-gated ion channel. These subunits play a key role in the plasticity of synapses, which is believed to underlie memory and learning.

Glutamate receptors are the predominant excitatory neurotransmitter receptors in the mammalian brain and are activated in a variety of normal neurophysiologic processes. The classification of glutamate receptors is based on their pharmacologic responses to different pharmacologic agonists. Thus, one class, the NMDA receptors, have N-methyl-D-aspartate as an agonist. NMDA receptor mRNA is expressed in neuronal cells throughout the brain, particularly in the hippocampus, cerebral cortex, and cerebellum.

Each NMDA receptor contains four or five subunits. The receptor oligomers are formed from members of three gene families. The subunits are called NR1 (sigma, not related to the sigma receptors), NR2 (epsilon) A-D, and NR3 A-C. Each subunit has an extracellular ligand binding domain, which links to the transmembrane ion pore. The most common arrangement contains two NR1 subunits that bind glycine and two NR2 that bind glutamate. GRIN1 is an example of an NR1 subunit. Five NMDA receptor subunits have now been characterized in both rat and mouse brains. A unique feature of NMDA receptor is the requirement for both (1) glutamate and (2) the co-agonist glycine for the efficient opening of the ion channel which is a part of this receptor. A third requirement is membrane depolarization.

NMDA receptor is modulated by a number of endogenous and exogenous compounds. Magnesium not only blocks the NMDA channel in a voltage-dependent manner but also potentiates NMDA-induced responses at positive membrane potentials. Na⁺, K⁺ and Ca²⁺ not only pass through the NMDA receptor channel, but also modulate the activity of NMDA receptors. Zn2+ blocks the NMDA current in a noncompetitive and a voltage-independent manner. Polyamines do not directly activate NMDA receptors, but instead act to potentiate or inhibit glutamate-mediated responses. The activity of NMDA receptors is also strikingly sensitive to the changes in H⁺ concentration, and partially inhibited by the ambient concentration of H⁺ under physiological conditions.

Nucleotide: “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Peripheral Nervous System (PNS): The part of an animal's nervous system other than the Central Nervous System. Generally, the PNS is located in the peripheral parts of the body and includes cranial nerves, spinal nerves and their branches, and the autonomic nervous system.

Pharmaceutically acceptable carriers: Remington's Pharmaceutical Sciences, by E. W. Martin, Mack Publishing Co., Easton, Pa., 15th Edition (1975), describes compositions and formulations suitable for pharmaceutical delivery of stem cells herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: Refers to a chemical compound, cells or composition capable of inducing a desired therapeutic or prophylactic effect when properly administered to a subject or a cell. “Incubating” includes exposing a target to an agent for a sufficient period of time for the agent to interact with a cell. “Contacting” includes incubating an agent in solid or in liquid form with a cell.

Polypeptide: A polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those which are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell. An “epitope” is a region of a polypeptide capable of binding an immunoglobulin generated in response to contact with an antigen. Thus, smaller peptides containing the biological activity of insulin, or conservative variants of the insulin, are thus included as being of use.

The term “soluble” refers to a form of a polypeptide that is not inserted into a cell membrane.

The term “substantially purified polypeptide” as used herein refers to a polypeptide which is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative substitutions replace one amino acid with another amino acid that is similar in size, hydrophobicity, etc. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Variations in the cDNA sequence that result in amino acid changes, whether conservative or not, are usually minimized in order to preserve the functional and immunologic identity of the encoded protein. The immunologic identity of the protein may be assessed by determining whether it is recognized by an antibody; a variant that is recognized by such an antibody is immunologically conserved. Any cDNA sequence variant will preferably introduce no more than twenty, and preferably fewer than ten amino acid substitutions into the encoded polypeptide. Variant amino acid sequences may, for example, be 80, 90 or even 95% or 98% identical to the native amino acid sequence. Programs and algorithms for determining percentage identity can be found at the NCBI website.

Precursor cell: A pluripotent cell that can generate a fully differentiated functional cell of at least one given cell type, or of more than one lineage. Generally, precursor cells can divide. After division, a precursor cell can remain a precursor cell, or may proceed to terminal differentiation. A “muscle precursor cell” is a precursor cell that can generate a fully differentiated functional muscle cell, such as a cardiomyocyte or a skeletal muscle cell. One specific, non-limiting example of a muscle precursor cell is a “cardiac precursor cell,” which is a cell that gives rise to cardiac muscle cells. A “neuronal precursor cell” is a precursor cell that can differentiate into a neuronal cell.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, for example, by genetic engineering techniques. Similarly, a recombinant protein is one encoded by a recombinant nucleic acid molecule.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of antibodies or antigen binding fragments, and the corresponding cDNA sequence, will possess a relatively high degree of sequence identity when aligned using standard methods. This homology will be more significant when the orthologous proteins or cDNAs are derived from species that are more closely related, compared to species more distantly related (for example, human and murine sequences).

Methods of alignment of sequences for comparison are well known in the art. Various programs and alignment algorithms are described in Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244 9, 1988); Higgins and Sharp, CABIOS 5:151-153, 1989; Corpet et al., Nuc. Acids Res. 16:10881-90, 1988; Huang et al., Computer Appls. in the Biosciences 8:155-65, 1992; and Pearson et al., Meth. Mol. Bio. 24:307-31, 1994. Altschul et al., J. Mol. Biol. 215:403-410, 1990, presents a detailed consideration of sequence alignment methods and homology calculations.

Skeletal-based precursor of cardiomyocytes (Spoc) cells: Pluripotent cells derived from skeletal muscle, which do not express the cell surface markers c-met or c-kit. As undetectable amounts of c-kit and c-met are expressed these cells, they also identified as c-kit⁻c-met⁻CD34⁻Sca-1⁻Pax(3/7)⁻ cells. Spoc cells can be differentiated into either cardiomyocytes or neurons. In one embodiment Spoc cells are muscle derived precursor cells that are about 4 μm to about 10 μM in diameter when cultured in vitro. These cells remain in suspension and proliferate when cultured in the presence of a growth factor. Specific, non-limiting examples of growth factors of use in propagating Spoc cell are FGF, EGF, or a combination thereof.

In one embodiment, Spoc cells differentiate into spontaneously beating cardiomyocytes in vitro. During a proliferative phase (for example, about 7 days after being maintained in vitro in the presence of a growth factor), Spoc cells cluster and increase in size to about 10-14 μm in diameter. The cells in these clusters, referred to as cardiac precursors from spoc (CS) cells, have the ability to differentiate into mature cardiac muscle cells when cultured in the absence of growth factors. Methods for isolating and differentiating Spoc cells are disclosed herein.

Spontaneous: Arising from an internal cause, resulting from internal or natural processes, with no apparent external influence. A “spontaneously beating cardiomyocyte” is a cell that begins to beat as a result of internal signals.

Stem cell: A cell that can generate a fully differentiated functional cell of more than one given cell type. A stem cell can be totipotent. The role of stem cells in vivo is to replace cells that are destroyed during the normal life of an animal. Generally, stem cells can divide without limit. After division, the stem cell may remain as a stem cell, become a precursor cell, or proceed to terminal differentiation. Although appearing morphologically unspecialized, the stem cell may be considered differentiated where the possibilities for further differentiation are limited. A “muscle stem cell” is a stem cell derived from muscle or that gives rise to muscle cells after differentiation. One specific, non-limiting example of a muscle stem cell is a cell that gives rise to cardiac muscle cells.

Subject: Any mammal, such as humans, non-human primates, pigs, sheep, cows, rodents and the like, which is to be the recipient of the particular treatment. In one embodiment, a subject is a human subject or a murine subject.

Suspension: A dispersion of solid particles, such as a cell, throughout the body of a liquid, such as a culture medium or an isotonic (physiologically compatible) buffer.

Synapse: Highly specialized intercellular junctions between neurons and between neurons and effector cells across which a nerve impulse is conducted (synaptically active). Generally, the nerve impulse is conducted by the release from one neuron (presynaptic neuron) of a chemical transmitter (such as dopamine or serotonin) which diffuses across the narrow intercellular space to the other neuron or effector cell (post-synaptic neuron). Generally neurotransmitters mediate their effects by interacting with specific receptors incorporated in the post-synaptic cell. “Synaptically active” refers to cells (for example, differentiated neurons) which receive and transmit action potentials characteristic of mature neurons.

Therapeutic agent: Used in a generic sense, it includes treating agents, prophylactic agents, and replacement agents.

Therapeutically effective amount: The amount of agent (including cells) that is sufficient to prevent, treat, reduce and/or ameliorate the symptoms and/or underlying causes of any of a disorder or disease. In one embodiment, a “therapeutically effective amount” is sufficient to reduce or eliminate a symptom of a neurologic disorder. In another embodiment, a therapeutically effective amount is an amount sufficient to overcome the disease itself.

A therapeutically effective amount of a cell can be administered in a single dose, or in several doses, for example daily, during a course of treatment. However, the effective amount of the cells will be dependent on the subject being treated, the severity and type of the condition, and the manner of administration of the compound. “Administering” can be accomplished by introducing the therapeutically effective amount locally or systemically into the subject. Systemic introduction can be accomplished by using an intravenous, intramuscular, transcutaneous or subcutaneous means. Such means could include introducing the therapeutically effective amount via injection, or via catheter. Local administration can be accomplished, for example, by direct injection into the affected area, or by implanting a substrate for controlled release.

The general term “administering a therapeutically effective amount to the subject” is understood to include all animals (for example, humans, apes, dogs, cats, horses, and cows) that have or may develop a disorder.

Transfected: A transfected cell is a cell into which has been introduced a nucleic acid molecule by molecular biology techniques. As used herein, the term transduction encompasses all techniques by which a nucleic acid molecule might be introduced into such a cell, including transduction with viral vectors, transformation with plasmid vectors, and introduction of DNA by electroporation, lipofection, and particle gun acceleration.

Transplantation: The transfer of a tissue or an organ, or a portion thereof, from one body or part of the body to another body or part of the body.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Spoc Cells

Stem cells derived from skeletal muscle (Spoc cells) have been isolated (see U.S. patent application Ser. No. 10/003,400, filed Oct. 22, 2001, and published PCT Publication No. WO 03/035838 A2, both of which are incorporated herein by reference in their entirety). Spoc cells do not express the cell surface markers c-met, c-kit, or CD34. Spoc cells can be further purified by sorting for the Sca-1 cell surface marker (found on hematopoietic cells). Spoc cells are thus Sca-1⁻, distinguishing them from side population (SP) cells. In addition, spoc cells do not express the Pax3 and Pax 7 transcription factors (Pax(3/7)). Thus, in one non-limiting example, spoc cells are c-kit⁻c-met⁻CD34⁻Sca-1⁻Pax(3/7)⁻.

Spoc cells can be isolated from any age mammal, either human or non-human. Thus Spoc cells can be obtained from an embryo, a fetus, a child or an adult of any mammalian species. A human or murine c-kit^{-c-met}⁻CD34⁻Sca-1⁻Pax(3/7)⁻ cell can be differentiated into a cardiomyocyte or a neuron in vitro. In one embodiment, the Spoc cell is between about 3 μm and about 10 μm in diameter. For example, the diameter of a Spoc cell can be from about 4 μm to about 10 μm, or is about 4 μm in diameter. Culture conditions for Spoc cells have been identified and are disclosed in U.S. patent application Ser. No. 10/003,400, filed Oct. 22, 2001, and published PCT Publication No. WO 03/035838 A2, both of which are incorporated herein by reference in their entirety.

Briefly, Spoc cells can be separated by size from a suspension of muscle cells and the cells are cultured on a solid substrate at 37° C. in the presence of CO₂. The cells that remain in suspension in the culture medium after about 7 days in the presence of a growth factor are isolated. This procedure is different than the serial preplating technique used to isolate myoblasts and satellite cells, wherein the quicker attachment of fibroblasts to a solid substrate, compared to myoblasts and satellite cells, is used to separate the myoblasts and satellite cells from the contaminating fibroblasts. Following a series of quick (15 minutes to one hour) preplating steps to remove the fibroblasts, the myoblasts or satellite cells are allowed to adhere to the solid substrate and the cells remaining in suspension (which would include the c-kit^{-c-met}⁻CD34⁻Sca-1⁻Pax(3/7)⁻ cells disclosed herein) are discarded.

Spoc cells can be obtained from the muscle of a subject. Muscle tissue can be prepared for the purpose of isolating or obtaining individual Spoc cells by using methods well known to one of skill in the art. Examples of methods of tissue preparation include enzymatic digestion with enzymes such as collagenase, mechanical disruption using instruments such as hand-held or motor-driven homogenizers, or by chemical disruption using, for example, chelators of calcium and magnesium.

Isolated Spoc cells can be maintained in culture. The Spoc cells can further be differentiated into cardiomyocytes or neurons. Spoc cells can be further identified by the expression of specific markers (see below). Culture conditions have been disclosed for differentiating Spoc cells into cardiomyocytes (see below and see U.S. patent application Ser. No. 10/003,400, filed Oct. 22, 2001, and published PCT Publication No. WO 03/035838 A2, both of which are incorporated herein by reference in their entirety).

The differentiation of Spoc cells into cardiomyocytes can be assessed by observing morphological changes. In some examples, differentiated Spoc cells are spontaneously beating cardiomyocytes. In several embodiments, organized gap junctions and sarcomeres with clear Z-lines and A- and I-bands, are observed in the differentiated Spoc cells. In addition, certain examples of the differentiated Spoc cells may be mono- or multi-nucleate. In one embodiment the cells are bi-nucleate. Similarly, the differentiation of Spoc cells, or a subset of Spoc cells, into neurons can be assessed by observing morphological changes. In some examples, expression of a neurologic marker such as β-tubulin, neuron specific enolase, a neurotransmitter, or an enzyme specific to neuronal cells can be assessed. In addition, it is possible to evaluate the differentiation of Spoc cells into neurons using electrophysiological means, such as measuring voltage or action potentials.

U.S. patent application Ser. No. 10/003,400, filed Oct. 22, 2001, and published PCT Application No. WO 03/035838 (both incorporated herein by reference) disclose a method for isolation of Spoc cells that includes obtaining the cells from the muscle of a subject. The isolated Spoc cell can be transduced using standard procedures known in molecular biology in order to introduce a nucleic acid molecule of interest into the cell. In one embodiment, the nucleic acid molecule encodes a polypeptide. The polypeptide encoded by the nucleic acid molecule can be from the same species as the cells (homologous), or can be from a different species (heterologous). For example, a nucleic acid molecule can be utilized that supplements or replaces deficient production of a peptide by the tissue of the host wherein such deficiency is a cause of the symptoms of a particular disorder. In this case, the cells act as a source of the peptide. In one specific, non-limiting example the polypeptide is the cardiac specific transcription factor GATA-4.

### Antibodies

Antibodies which specifically bind to Spoc cells, a sub-population of Spoc cells, or neuronal precursor cells can be produced. In one embodiment, the antibody specifically binds all Spoc cells. In another embodiment the antibody specifically binds a sub-population of Spoc cells. Disclosed herein are antibodies that specifically binds a sub-population of Spoc cells that are neuronal precursor cells. In one example, the antibody specifically binds a mammalian c-kit^{-c-met}⁻CD34⁻Sca-1⁻Pax(3/7)⁻ from any age mammal, either human or non-human. Thus the antibody binds Spoc cells, a sub-population of Spoc cells, or neuronal precursor cells from an embryo, a fetus, a child, or an adult of any mammalian species. In one embodiment, the antibody binds a mammalian Spoc cell, such as a human, porcine, or murine c-kit⁻c-met⁻CD34⁻Sca-1⁻Pax(3/7)⁻ cell.

It is disclosed herein that a subset, or a sub-population, of Spoc cells express a GRIN1 polypeptide. Thus, antibodies that bind at least eight consecutive amino acids of a GRIN1 polypeptide can be used to identify a specific sub-population of cells, such as a sub-population of Spoc cells. More specifically, the antibodies that bind at least eight consecutive amino acids of a GRIN1 polypeptide can be used to identify cells, for example a sub-population of Spoc cells, or neuronal precursor cells, that can differentiate into neuronal cells.

The amino acid sequence of GRIN1, and splice variants of GRIN1 from a variety of mammalian species are known in the art. For example, one form of murine GRIN1 has the amino acid sequence (SEQ ID NO: 1):

MSTMHLLTFA LLFSCSFARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYNW
NHIILLVSDD HEGRAAQKRL ETLLEERESK AEKVLQFDPG
TKNVTALLME ARDLEARVII LSASEDDAAT VYRAAAMLNM
TGSGYVWLVG EREISGNALR YAPDGIIGLQ LINGKNESAH
ISDAVGVVAQ AVHELLEKEN ITDPPRGCVG NTNIWKTGPL
FKRVLMSSKY ADGVTGRVEF NEDGDRKFAN YSIMNLQNRK
LVQVGIYNGT HVIPNDRKII WPGGETEKPR GYQMSTRLKI
VTIHQEPFVY VKPTMSDGTC KEEFTVNGDP VKKVICTGPN
DTSPGSPRHT VPQCCYGFCV DLLIKLARTM NFTYEVHLVA
DGKFGTQERV NNSNKKEWNG MMGELLSGQA DMIVAPLTIN
NERAQYIEFS KPFKYQGLTI LVKKEIPRST LDSFMQPFQS
TLWLLVGLSV HVVAVMLYLL DRFSPFGRFK VNSEEEEEDA
LTLSSAMWFS WGVLLNSGIG EGAPRSFSAR ILGMVWAGFA
MIIVASYTAN LAAFLVLDRP EERITGINDP RLRNPSDKFI
YATVKQSSVD IYFRRQVELS TMYRHMEKHN YESAAEAIQA
VRDNKLHAFI WDSAVLEFEA SQKCDLVTTG ELFFRSGFGI
GMRKDSPWKQ NVSLSILKSH ENGFMEDLDK TWVRYQECDS
RSNAPATLTF ENMAGVFMLV AGGIVAGIFL IFIEIAYKRH
KDARRKQMQL AFAAVNVWRK NLQQYHPTDI TGPLNLSDPS
VSTVV

Another form of murine GRIN1 has the amino acid sequence (SEQ ID NO: 2):

MSTMHLLTFA LLFSCSFARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYNW
NHIILLVSDD HEGRAAQKRL ETLLEERESK AEKVLQFDPG
TKNVTALLME ARDLEARVII LSASEDDAAT VYRAAAMLNM
TGSGYVWLVG EREISGNALR YAPDGIIGLQ LINGKNESAH
ISDAVGVVAQ AVHELLEKEN ITDPPRGCVG NTNIWKTGPL
FKRVLMSSKY ADGVTGRVEF NEDGDRKFAN YSIMNLQNRK
LVQVGIYNGT HVIPNDRKII WPGGETEKPR GYQMSTRLKI
VTIHQEPFVY VKPTMSDGTC KEEFTVNGDP VKKVICTGPN
DTSPGSPRHT VPQCCYGFCV DLLIKLARTM NFTYEVHLVA
DGKFGTQERV NNSNKKEWNG MMGELLSGQA DMIVAPLTIN
NERAQYIEFS KPFKYQGLTI LVKKEIPRST LDSFMQPFQS
TLWLLVGLSV HVVAVMLYLL DRFSPFGRFK VNSEEEEEDA
LTLSSAMWFS WGVLLNSGIG EGAPRSFSAR ILGMVWAGFA
MIIVASYTAN LAAFLVLDRP EERITGINDP RLRNPSDKFI
YATVKQSSVD IYFRRQVELS TMYRHMEKHN YESAAEAIQA
VRDNKLHAFI WDSAVLEFEA SQKCDLVTTG ELFFRSGFGI
GMRKDSPWKQ NVSLSILKSH ENGFMEDLDK TWVRYQECDS
RSNAPATLTF ENMAGVFMLV AGGIVAGIFL IFIEIAYKRH
KDARRKQMQL AFAAVNVWRK NLQDRKSGRA EPDPKKKATF
RAITSTLASS FKRRRSSKDT STGGGRGALQ NQKDTVLPRR
AIEREEGQLQ LCSRHRES

Several isoforms of rat GRIN1 are known to the art. For example, the rat NMDAR1-1a subunit has the following sequence (SEQ ID NO: 3):

MSTMHLLTFA LLFSCSFARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYNW
NHIILLVSDD HEGRAAQKRL ETLLEERESK AEKVLQFDPG
TKNVTALLME ARELEARVII LSASEDDAAT VYRAAAMLNM
TGSGYVWLVG EREISGNALR YAPDGIIGLQ LINGKNESAH
ISDAVGVVAQ AVHELLEKEN ITDPPRGCVG NTNIWKTGPL
FKRVLMSSKY ADGVTGRVEF NEDGDRKFAN YSIMNLQNRK
LVQVGIYNGT HVIPNDRKII WPGGETEKPR GYQMSTRLKI
VTIHQEPFVY VKPTMSDGTC KEEFTVNGDP VKKVICTGPN
DTSPGSPRHT VPQCCYGFCI DLLIKLARTM NFTYEVHLVA
DGKFGTQERV NNSNKKEWNG MMGELLSGQA DMIVAPLTIN
NERAQYIEFS KPFKYQGLTI LVKKEIPRST LDSFMQPFQS
TLWLLVGLSV HVVAVMLYLL DRFSPFGRFK VNSEEEEEDA
LTLSSAMWFS WGVLLNSGIG EGAPRSFSAR ILGMVWAGFA
MIIVASYTAN LAAFLVLDRP EERITGINDP RLRNPSDKFI
YATVKQSSVD IYFRRQVELS TMYRHMEKHN YESAAEAIQA
VRDNKLHAFI WDSAVLEFEA SQKCDLVTTG ELFFRSGFGI
GMRKDSPWKQ NVSLSILKSH ENGFMEDLDK TWVRYQECDS
RSNAPATLTF ENMAGVFMLV AGGIVAGIFL IFIEIAYKRH
KDARRKQMQL AFAAVNVWRK NLQDRKSGRA EPDPKKKATF
RAITSTLASS FKRRRSSKDT STGGGRGALQ NQKDTVLPRR
AIEREEGQLQ LCSRHRES

The rat NMDAR1-1b subunit has the following sequence (SEQ ID NO: 4):

MSTMHLLTFA LLFSCSFARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYNW
NHIILLVSDD HEGRAAQKRL ETLLEERESK SKKRNYENLD
QLSYDNKRGP KAEKVLQFDP GTKNVTALLM EARELEARVI
ILSASEDDAA TVYRAAAMLN MTGSGYVWLV GEREISGNAL
RYAPDGIIGL QLINGKNESA HISDAVGVVA QAVHELLEKE
NITDPPRGCV GNTNIWKTGP LFKRVLMSSK YADGVTGRVE
FNEDGDRKFA NYSIMNLQNR KLVQVGIYNG THVIPNDRKI
IWPGGETEKP RGYQMSTRLK IVTIHQEPFV YVKPTMSDGT
CKEEFTVNGD PVKKVICTGP NDTSPGSPRH TVPQCCYGFC
IDLLIKLART MNFTYEVHLV ADGKFGTQER VNNSNKKEWN
GMMGELLSGQ ADMIVAPLTI NNERAQYIEF SKPFKYQGLT
ILVKKEIPRS TLDSFMQPFQ STLWLLVGLS VHVVAVMLYL
LDRFSPFGRF KVNSEEEEED ALTLSSAMWF SWGVLLNSGI
GEGAPRSFSA RILGMVWAGF AMIIVASYTA NLAAFLVLDR
PEERITGIND PRLRNPSDKF IYATVKQSSV DIYFRRQVEL
STMYRHMEKH NYESAAEAIQ AVRDNKLHAF IWDSAVLEFE
ASQKCDLVTT GELFFRSGFG IGMRKDSPWK QNVSLSILKS
HENGFMEDLD KTWVRYQECD SRSNAPATLT FENMAGVFML
VAGGIVAGIF LIFIEIAYKR HKDARRKQMQ LAFAAVNVWR
KNLQDRKSGR AEPDPKKKAT FRAITSTLAS SFKRRRSSKD
TSTGGGRGAL QNQKDTVLPR RAIEREEGQL QLCSRHRES

The rat NMDAR1-2a subunit has the following sequence (SEQ ID NO: 5):

MSTMHLLTFA LLFSCSFARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYNW
NHIILLVSDD HEGRAAQKRL ETLLEERESK AEKVLQFDPG
TKNVTALLME ARELEARVII LSASEDDAAT VYRAAAMLNM
TGSGYVWLVG EREISGNALR YAPDGIIGLQ LINGKNESAH
ISDAVGVVAQ AVHELLEKEN ITDPPRGCVG NTNIWKTGPL
FKRVLMSSKY ADGVTGRVEF NEDGDRKFAN YSIMNLQNRK
LVQVGIYNGT HVIPNDRKII WPGGETEKPR GYQMSTRLKI
VTIHQEPFVY VKPTMSDGTC KEEFTVNGDP VKKVICTGPN
DTSPGSPRHT VPQCCYGFCI DLLIKLARTM NFTYEVHLVA
DGKFGTQERV NNSNKKEWNG MMGELLSGQA DMIVAPLTIN
NERAQYIEFS KPFKYQGLTI LVKKEIPRST LDSFMQPFQS
TLWLLVGLSV HVVAVMLYLL DRFSPFGRFK VNSEEEEEDA
LTLSSAMWFS WGVLLNSGIG EGAPRSFSAR ILGMVWAGFA
MIIVASYTAN LAAFLVLDRP EERITGINDP RLRNPSDKFI
YATVKQSSVD IYFRRQVELS TMYRHMEKHN YESAAEAIQA
VRDNKLHAFI WDSAVLEFEA SQKCDLVTTG ELFFRSGFGI
GMRKDSPWKQ NVSLSILKSH ENGFMEDLDK TWVRYQECDS
RSNAPATLTF ENMAGVFMLV AGGIVAGIFL IFIEIAYKRH
KDARRKQMQL AFAAVNVWRK NLQSTGGGRG ALQNQKDTVL
PRRAIEREEG QLQLCSRHRE S

The rat NMDAR1-2b subunit has the following sequence (SEQ ID NO: 6):

MSTMHLLTFA LLFSCSFARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYNW
NHIILLVSDD HEGRAAQKRL ETLLEERESK SKKRNYENLD
QLSYDNKRGP KAEKVLQFDP GTKNVTALLM EARELEARVI
ILSASEDDAA TVYRAAAMLN MTGSGYVWLV GEREIEGNAL
RYAPDGIIGL QLINGKNESA HISDAVGVVA QAVHELLEKE
NITDPPRGCV GNTNIWKTGP LFKRVLMSSK YADGVTGRVE
FNEDGDRKFA NYSIMNLQNR KLVQVGIYNG THVIPNDRKI
IWPGGETEKP RGYQMSTRLK IVTIHQEPFV YVKPTMSDGT
CKEEFTVNGD PVKKVICTGP NDTSPGSPRH TVPQCCYGFC
IDLLIKLART MNFTYEVHLV ADGKFGTQER VNNSNKKEWN
GMMGELLSGQ ADMIVAPLTI NNERAQYIEF SKPFKYQGLT
ILVKKEIPRS TLDSFMQPFQ STLWLLVGLS VHVVAVMLYL
LDRFSPFGRF KVNSEEEEED ALTLSSAMWF SWGVLLNSGI
GEGAPRSFSA RILGMVWAGF AMIIVASYTA NLAAFLVLDR
PEERITGIND PRLRNPSDKF IYATVKQSSV DIYFRRQVEL
STMYRHMEKH NYESAAEAIQ AVRDNKLHAF IWDSAVLEFE
ASQKCDLVTT GELFFRSGFG IGMRKDSPWK QNVSLSILKS
HENGFMEDLD KTWVRYQECD SRSNAPATLT FENMAGVFML
VAGGIVAGIF LIFIEIAYKR HKDARRKQMQ LAFAAVNVWR
KNLQSTGGGR GALQNQKDTV LPRRAIEREE GQLQLCSRHR
ES

The rat NMDAR1-3a subunit has the following sequence (SEQ ID NO: 7):

MSTMHLLTFA LLFSCSFARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYNW
NHIILLVSDD HEGRAAQKRL ETLLEERESK AEKVLQFDPG
TKNVTALLME ARELEARVII LSASEDDAAT VYRAAANLNM
TGSGYVWLVG EREISGNALR YAPDGIIGLQ LINGKNESAH
ISDAVGVVAQ AVHELLEKEN ITDPPRGCVG NTNIWKTGPL
FKRVLMSSKY ADGVTGRVEF NEDGDRKFAN YSIMNLQNRK
LVQVGIYNGT HVIPNDRKII WPGGETEKPR GYQMSTRLKI
VTIHQEPFVY VKPTMSDGTC KEEFTVNGDP VKKVICTGPN
DTSPGSPRHT VPQCCYGFCI DLLIKLARTM NFTYEVHLVA
DGKFGTQERV NNSNKKEWNG MMGELLSGQA DMIVAPLTIN
NERAQYIEFS KPFKYQGLTI LVKKEIPRST LDSFMQPFQS
TLWLLVGLSV HVVAVMLYLL DRFSPFGRFK VNSEEEEEDA
LTLSSANWFS WGVLLNSGIG EGAPRSFSAR ILGMVWAGFA
MIIVASYTAN LAAFLVLDRP EERITGINDP RLRNPSDKFI
YATVKQSSVD IYFRRQVELS TMYRHMEKHN YESAAEAIQA
VRDNKLHAFI WDSAVLEFEA SQKCDLVTTG ELFFRSGFGI
GMRKDSPWKQ NVSLSILKSH ENGFMEDLDK TWVRYQECDS
RSNAPATLTF ENMAGVFMLV AGGIVAGIFL IFIEIAYKRH
KDARRKQMQL AFAAVNVWRK NLQDRKSGRA EPDPKKKATF
RAITETLASS FKRRRSSKDT QYHPTDITGP LNLSDPSVST
VV

The rat NMDAR1-3b subunit has the following sequence (SEQ ID NO: 8):

MSTMHLLTFA LLFSCSFARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYNW
NHIILLVSDD HEGRAAQKRL ETLLEERESK SKKRNYENLD
QLSYDNKRGP KAEKVLQFDP GTKNVTALLM EARELEARVI
ILSASEDDAA TVYRAAAMLN MTGSGYVWLV GEREISGNAL
RYAPDGIIGL QLINGKNESA HISDAVGVVA QAVHELLEKE
NITDPPRGCV GNTNIWKTGP LFKRVLMSSK YADGVTGRVE
FNEDGDRKFA NYSIMNLQNR KLVQVGIYNG THVIPNDRKI
IWPGGETEKP RGYQMSTRLK IVTIHQEPFV YVKPTMSDGT
CKEEFTVNGD PVKKVICTGP NDTSPGSPRH TVPQCCYGFC
IDLLIKLART MNFTYEVHLV ADGKFGTQER VNNSNKKEWN
GMMGELLSGQ ADMIVAPLTI NNERAQYIEF SKPFKYQGLT
ILVKKEIPRS TLDSFMQPFQ STLWLLVGLS VHVVAVMLYL
LDRFSPFGRF KVNSEEEEED ALTLSSAMWF SWGVLLNSGI
GEGAPRSFSA RILGMVWAGF AMIIVASYTA NLAAFLVLDR
PEERITGIND PRLRNPSDKF IYATVKQSSV DIYFRRQVEL
STMYRHMEKH NYESAAEAIQ AVRDNKLHAF IWDSAVLEFE
ASQKCDLVTT GELFFRSGFG IGMRKDSPWK QNVSLSILKS
HENGFMEDLD KTWVRYQECD SRSNAPATLT FENMAGVFML
VAGGIVAGIF LIFIEIAYKR HKDARRKQMQ LAFAAVNVWR
KNLQDRKSGR AEPDPKKKAT FRAITSTLAS SFKRRRSSKD
TQYHPTDITG PLNLSDPSVS TVV

The rat NMDAR1-4a subunit has the following sequence (SEQ ID NO: 9):

MSTMHLLTFA LLFSCSFARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYNW
NHIILLVSDD HEGRAAQKRL ETLLEERESK AEKVLQFDPG
TKNVTALLME ARELEARVII LSASEDDAAT VYRAAAMLNM
TGSGYVWLVG EREISGNALR YAPDGIIGLQ LINGKNESAH
ISDAVGVVAQ AVHELLEKEN ITDPPRGCVG NTNIWKTGPL
FKRVLMSSKY ADGVTGRVEF NEDGDRKFAN YSIMNLQNRK
LVQVGIYNGT HVIPNDRKII WPGGETEKPR GYQMSTRLKI
VTIHQEPFVY VKPTMSDGTC KEEFTVNGDP VKKVICTGPN
DTSPGSPRHT VPQCCYGFCI DLLIKLARTM NFTYEVHLVA
DGKFGTQERV NNSNKKEWNG MMGELLSGQA DMIVAPLTIN
NERAQYIEFS KPFKYQGLTI LVKKEIPRST LDSFMQPFQS
TLWLLVGLSV HVVAVMLYLL DRFSPFGRFK VNSEEEEEDA
LTLSSAMWFS WGVLLNSGIG EGAPRSFSAR ILGMVWAGFA
MIIVASYTAN LAAFLVLDRP EERITGINDP RLRNPSDKFI
YATVKQSSVD IYFRRQVELS TMYRHMEKHN YESAAEAIQA
VRDNKLHAFI WDSAVLEFEA SQKCDLVTTG ELFFRSGFGI
GMRKDSPWKQ NVSLSILKSH ENGFMEDLDK TWVRYQECDS
RSNAPATLTF ENMAGVFMLV AGGIVAGIFL IFIEIAYKRH
KDARRKQMQL AFAAVNVWRK NLQQYHPTDI TGPLNLSDPS
VSTVV

The rat NMDAR1-4b subunit has the following sequence (SEQ ID NO: 10):

MSTMHLLTFA LLFSCSFARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYNW
NHIILLVSDD HEGRAAQKRL ETLLEERESK SKKRNYENLD
QLSYDNKRGP KAEKVLQFDP GTKNVTALLM EARELEARVI
ILSASEDDAA TVYRAAAMLN MTGSGYVWLV GEREISGNAL
RYAPDGIIGL QLINGKNESA HISDAVGVVA QAVHELLEKE
NITDPPRGCV GNTNIWKTGP LFKRVLMSSK YADGVTGRVE
FNEDGDRKFA NYSIMNLQNR KLVQVGIYNG THVIPNDRKI
IWPGGETEKP RGYQMSTRLK IVTIHQEPFV YVKPTMSDGT
CKEEFTVNGD PVKKVICTGP NDTSPGSPRH TVPQCCYGFC
IDLLIKLART MNFTYEVHLV ADGKFGTQER VNNSNKKEWN
GMMGELLSGQ ADMIVAPLTI NNERAQYIEF SKPFKYQGLT
ILVKKEIPRS TLDSFMQPFQ STLWLLVGLS VHVVAVMLYL
LDRFSPFGRF KVNSEEEEED ALTLSSAMWF SWGVLLNSGI
GEGAPRSFSA RILGMVWAGF AMIIVASYTA NLAAFLVLDR
PEERITGIND PRLRNPSDKF IYATVKQSSV DIYFRRQVEL
STMYRHMEKH NYESAAEAIQ AVRDNKLHAF IWDSAVLEFE
ASQKCDLVTT GELFFRSGFG IGMRKDSPWK QNVSLSILKS
HENGFMEDLD KTWVRYQECD SRSNAPATLT FENMAGVFML
VAGGIVAGIF LIFIEIAYKR HKDARRKQMQ LAFAAVNVWR
KNLQQYHPTD ITGPLNLSDP SVSTVV

One form of human GRIN1 has the following amino acid sequence (SEQ ID NO: 11):

MSTMRLLTLA LLFSCSVARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYSW
NHIILLVSDD HEGRAAQKRL ETLLEERESK AEKVLQFDPG
TKNVTALLME AKELEARVII LSASEDDAAT VYRAAAMLNM
TGSGYVWLVG EREISGNALR YAPDGILGLQ LINGKNESAH
ISDAVGVVAQ AVHELLEKEN ITDPPRGCVG NTNIWKTGPL
FKRVLMSSKY ADGVTGRVEF NEDGDRKFAN YSIMNLQNRK
LVQVGIYNGT HVIPNDRKII WPGGETEKPR GYQMSTRLKI
VTIHQEPFVY VKPTLSDGTC KEEFTVNGDP VKKVICTGPN
DTSPGSPRHT VPQCCYGFCI DLLIKLARTM NFTYEVHLVA
DGKFGTQERV NNSNKKEWNG MMGELLSGQA DMIVAPLTIN
NERAQYIEFS KPFKYQGLTI LVKKEIPRST LDSFMQPFQS
TLWLLVGLSV HVVAVMLYLL DRFSPFGRFK VNSEEEEEDA
LTLSSAMWFS WGVLLNSGIG EGAPRSFSAR ILGMVWAGFA
MIIVASYTAN LAAFLVLDRP EERITGINDP RLRNPSDKFI
YATVKQSSVD IYFRRQVELS TMYRHMEKHN YESAAEAIQA
VRDNKLHAFI WDSAVLEFEA SQKCDLVTTG ELFFRSGFGI
GMRKDSPWKQ NVSLSILKSH ENGFMEDLDK TWVRYQECDS
RSNAPATLTF ENMAGVFMLV AGGIVAGIFL IFIEIAYKRH
KDARRKQMQL AFAAVNVWRK NLQDRKSGRA EPDPKKKATF
RAITSTLASS FKRRRSSKDT STGGGRGALQ NQKDTVLPRR
AIEREEGQLQ LCSRHRES

There are three known isoforms (splice variants) of human GRIN1 (NR1-1, NR1-2, and NR1-3). NR1-1 has the following amino acid sequence (SEQ ID NO: 12):

MSTMRLLTLA LLFSCSVARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYSW
NHIILLVSDD HEGRAAQKRL ETLLEERESK AEKVLQFDPG
TKNVTALLME AKELEARVII LSASEDDAAT VYRAAAMLNM
TGSGYVWLVG EREISGNALR YAPDGILGLQ LINGKNESAH
ISDAVGVVAQ AVHELLEKEN ITDPPRGCVG NTNIWKTGPL
FKRVLMSSKY ADGVTGRVEF NEDGDRKFAN YSIMNLQNRK
LVQVGIYNGT HVIPNDRKII WPGGETEKPR GYQMSTRLKI
VTIHQEPFVY VKPTLSDGTC KEEFTVNGDP VKKVICTGPN
DTSPGSPRHT VPQCCYGFCI DLLIKLARTM NFTYEVHLVA
DGKFGTQERV NNSNKKEWNG MMGELLSGQA DMIVAPLTIN
NERAQYIEFS KPFKYQGLTI LVKKEIPRST LDSFMQPFQS
TLWLLVGLSV HVVAVMLYLL DRFSPFGRFK VNSEEEEEDA
LTLSSAMWFS WGVLLNSGIG EGAPRSFSAR ILGMVWAGFA
MIIVASYTAN LAAFLVLDRP EERITGINDP RLRNPSDKFI
YATVKQSSVD IYFRRQVELS TMYRHMEKHN YESAAEAIQA
VRDNKLHAFI WDSAVLEFEA SQKCDLVTTG ELFFRSGFGI
GMRKDSPWKQ NVSLSILKSH ENGFMEDLDK TWVRYQECDS
RSNAPATLTF ENMAGVFMLV AGGIVAGIFL IFIEIAYKRH
KDARRKQMQL AFAAVNVWRK NLQQYHPTDI TGPLNLSDPS
VSTVV

Human NR1-2 has the following amino acid sequence (SEQ ID NO: 13):

MSTMRLLTLA LLFSCSVARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSINLSFLRT VPPYSHQSSV WFEMMRVYSW
NHIILLVSDD HEGRAAQKRL ETLLEERESK AEKVLQFDPG
TKNVTALLME AKELEARVII LSASEDDAAT VYRAAAMLNM
TGSGYVWLVG EREISGNALR YAPDGILGLQ LINGKNESAH
ISDAVGVVAQ AVHELLEKEN ITDPPRGCVG NTNIWKTGPL
FKRVLMSSKY ADGVTGRVEF NEDGDRKFAN YSIMNLQNRK
LVQVGIYNGT HVIPNDRKII WPGGETEKPR GYQMSTRLKI
VTIHQEPFVY VKPTLSDGTC KEEFTVNGDP VKKVICTGPN
DTSPGSPRHT VPQCCYGFCI DLLIKLARTM NFTYEVHLVA
DGKFGTQERV NNSNKKEWNG MMGELLSGQA DMIVAPLTIN
NERAQYIEFS KPFKYQGLTI LVKKEIPRST LDSFMQPFQS
TLWLLVGLSV HVVAVMLYLL DRFSPFGRFK VNSEEEEEDA
LTLSSAMWFS WGVLLNSGIG EGAPRSFSAR ILGMVWAGFA
MIIVASYTAN LAAFLVLDRP EERITGINDP RLRNPSDKFI
YATVKQSSVD IYFRRQVELS TMYRHMEKHN YESAAEAIQA
VRDNKLHAFI WDSAVLEFEA SQKCDLVTTG ELFFRSGFGI
GMRKDSPWKQ NVSLSILKSH ENGFMEDLDK TWVRYQECDS
RSNAPATLTF ENMAGVFMLV AGGIVAGIFL IFIEIAYKRH
KDARRKQMQL AFAAVNVWRK NLQSTGGGRG ALQNQKDTVL
PRRAIEREEG QLQLCSRHRE S

Human NR1-3 has the following amino acid sequence (SEQ ID NO: 14):

MSTMRLLTLA LLFSCSVARA ACDPKIVNIG AVLSTRKHEQ
MFREAVNQAN KRHGSWKIQL NATSVTHKPN AIQMALSVCE
DLISSQVYAI LVSHPPTPND HFTPTPVSYT AGFYRIPVLG
LTTRMSIYSD KSIHLSFLRT VPPYSHQSSV WFEMMRVYSW
NHIILLVSDD HEGRAAQKRL ETLLEERESK AEKVLQFDPG
TKNVTALLME AKELEARVII LSASEDDAAT VYRAAAMLNM
TGSGYVWLVG EREISGNALR YAPDGILGLQ LINGKNESAH
ISDAVGVVAQ AVHELLEKEN ITDPPRGCVG NTNIWKTGPL
FKRVLMSSKY ADGVTGRVEF NEDGDRKFAN YSIMNLQNRK
LVQVGIYNGT HVIPNDRKII WPGGETEKPR GYQMSTRLKI
VTIHQEPFVY VKPTLSDGTC KEEFTVNGDP VKKVICTGPN
DTSPGSPRHT VPQCCYGFCI DLLIKLARTM NFTYEVHLVA
DGKFGTQERV NNSNKKEWNG MMGELLSGQA DMIVAPLTIN
NERAQYIEFS KPFKYQGLTI LVKKEIPRST LDSFMQPFQS
TLWLLVGLSV HVVAVMLYLL DRFSPFGRFK VNSEEEEEDA
LTLSSAMWFS WGVLLNSGIG EGAPRSFSAR ILGMVWAGFA
MIIVASYTAN LAAFLVLDRP EERITGINDP RLRNPSDKFI
YATVKQSSVD IYFRRQVELS TMYRHMEKHN YESAAEAIQA
VRDNKLHAFI WDSAVLEFEA SQKCDLVTTG ELFFRSGFGI
GMRKDSPWKQ NVSLSILKSH ENGFMEDLDK TWVRYQECDS
RSNAPATLTF ENNAGVFMLV AGGIVAGIFL IFIEIAYKRH
KDARRKQMQL AFAAVNVWRK NLQDRKSGRA EPDPKKKATF
RAITSTLASS FKRRRSSKDT STGGGRGALQ NQKDTVLPRR
AIEREEGQLQ LCSRHRES

Antibodies that bind a sub-population of Spoc cells and identify and/or isolate a neuronal precursor cell can bind to at least eight consecutive amino acids of a polypeptide having an amino acid sequence set forth as SEQ ID NO: 1. Alternatively, the antibodies that bind a sub-population of Spoc cells, or a neuronal precursor cell, can bind to at least eight consecutive amino acids of a polypeptide having an amino acid sequence set forth as SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 1, SEQ ID NO: 12, SEQ ID NO: 13, or SEQ ID NO: 14. In some embodiments, the antibodies that bind a sub-population of Spoc cells, or a neuronal precursor cell can bind at least one of the following GRIN1 peptides: KYADGVTGRV (SEQ ID NO: 15), KVLQFDPGTKN (SEQ ID NO: 16), KHNYESAAEAIQAVRD (SEQ ID NO: 17), or KIVNIGAVLSTRK (SEQ ID NO: 18).

The antibodies that bind a sub-population of Spoc cells and that are used to identify and/or isolate a neuronal precursor cell also can bind a polypeptide with an amino acid sequence at least 85%, at least 90%, at least 95%, at least 97%, at least 98%, or at least 99% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 1, SEQ ID NO: 12, SEQ ID NO: 13, or SEQ ID NO: 14.

Antibodies that bind a sub-population of Spoc cells and identify and/or isolate a neuronal precursor cell can bind to a full-length GRIN1 polypeptide, or a splice variant of the full-length GRIN1 polypeptide. Alternatively, the antibodies can bind a GRIN1 polypeptide having a mutation or variation in a post-translational modification, compared to a wildtype GRIN1 polypeptide, wherein the mutation or the alteration in the post-translational modification does not affect the immunogenic epitope recognized by the antibodies. Mutants of GRIN1 include GRIN1 polypeptides with a point mutation, an addition, or a deletion of the GRIN1 amino acid sequence, compared to the wildtype GRIN1 amino acid sequence. GRIN1 polypeptides with altered post-translational modifications include alterations in glycosylation, phosphorylation, acylation, amidation, methylation, sulfation, prenylation, and the like, compared to the wildtype GRIN1 polypeptide.

In one embodiment, an antibody that binds a sub-population of Spoc cells and that identifies and/or isolates a neuronal precursor cell is the 804 monoclonal antibody. Thus, the 804 monoclonal antibody can be used to identify a specific subpopulation of Spoc cells, specifically those Spoc cells that can differentiate into neuronal cells. This antibody was deposited with American Type Culture Collection in accordance with the Budapest Treaty on Mar. 24, 2004, ATCC Deposit No. PTA-5888.

Polyclonal antibodies, antibodies which consist essentially of pooled monoclonal antibodies with different epitopic specificities, as well as distinct monoclonal antibody preparations are included.

The preparation of polyclonal antibodies is well known to those skilled in the art. See, for example, Green et al., “Production of Polyclonal Antisera,” in: Immunochemical Protocols pages 1-5, Manson, ed., Humana Press 1992; Coligan et al., “Production of Polyclonal Antisera in Rabbits, Rats, Mice and Hamsters,” in: Current Protocols in Immunology, section 2.4.1, 1992.

The preparation of monoclonal antibodies likewise is conventional. See, for example, Kohler & Milstein, Nature 256:495, 1975; Coligan et al., sections 2.5.1-2.6.7; and Harlow et al., in: Antibodies: a Laboratory Manual, page 726, Cold Spring Harbor Pub., 1988. Briefly, monoclonal antibodies can be obtained by injecting mice with a composition comprising an antigen or a cell of interest, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, for example, Coligan et al., sections 2.7.1-2.7.12 and sections 2.9.1-2.9.3; Barnes et al., “Purification of Immunoglobulin G (IgG),” in: Methods in Molecular Biology, Vol. 10, pages 79-104, Humana Press, 1992.

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, for example, syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Antibodies can also be derived from a subhuman primate antibody. General techniques for raising therapeutically useful antibodies in baboons can be found, for example, in PCT Publication No. WO 91/11465, 1991, and Losman et al., Int. J. Cancer 46:310, 1990.

Alternatively, an antibody that specifically binds a Spoc cell or a sub-population of Spoc cells, such as neuronal precursor cells, can be derived from a humanized monoclonal antibody. Humanized monoclonal antibodies are produced by transferring mouse complementarity determining regions (CDRs) from heavy and light variable chains of the mouse immunoglobulin into a human variable domain, and then substituting some human residues in the framework regions with the murine counterparts. In addition, murine residues in the CDRs can be substituted with the human counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Nat'l Acad. Sci. U.S.A. 89:4285, 1992; Sandhu, Crit. Rev. Biotech. 12:437, 1992; and Singer et al., J. Immunol. 150:2844, 1993.

Antibodies can be derived from human antibody fragments isolated from a combinatorial immunoglobulin library. See, for example, Barbas et al., in: Methods: a Companion to Methods in Enzymology, Vol. 2, page 119, 1991; Winter et al., Ann. Rev. Immunol. 12:433, 1994. Cloning and expression vectors that are useful for producing a human immunoglobulin phage library can be obtained, for example, from STRATAGENE Cloning Systems (La Jolla, Calif.).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13, 1994; Lonberg et al., Nature 368:856, 1994; and Taylor et al., Int. Immunol. 6:579, 1994.

Antibodies include intact molecules as well as fragments thereof, such as Fab, F(ab′)₂, and Fv which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (SCA), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains, and usually have specific three-dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al., supra at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent (Inbar et al., Proc. Nat'l Acad. Sci. U.S.A. 69:2659, 1972). Alternatively, the variable chains can be linked by an intermolecular disulfide bond or cross-linked by chemicals such as glutaraldehyde. See, for example, Sandhu, supra. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: a Companion to Methods in Enzymology, Vol. 2, page 106, 1991).

Antibodies can be prepared using an intact cell, or intact polypeptide or fragments containing small peptides of interest isolated from Spoc cells as the immunizing antigen. The polypeptide or a peptide used to immunize an animal can be derived from substantially purified polypeptide, a polypeptide produced in host cells, in vitro translated cDNA, or chemical synthesis which can be conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (for example, a mouse, a rat, or a rabbit).

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (see for example, Coligan et al., Unit 9, Current Protocols in Immunology, Wiley Interscience, 1991).

It is also possible to use the anti-idiotype technology to produce monoclonal antibodies which mimic an epitope. For example, an anti-idiotypic monoclonal antibody made to a first monoclonal antibody will have a binding domain in the hypervariable region that is the “image” of the epitope bound by the first monoclonal antibody.

Binding affinity for a target antigen is typically measured or determined by standard antibody-antigen assays, such as competitive assays, saturation assays, or immunoassays such as ELISA or RIA. Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is, for example <1 μM, <100 nM, or <0.1 nM. Antibody molecules will typically have a K_{D} in the lower ranges. K_{D}=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible noncovalent associations such as electrostatic attraction, Van der Waals forces and hydrogen bonds.

Effector molecules, for example, therapeutic, diagnostic, or detection moieties can be linked to an antibody that specifically binds a Spoc cell, using any number of means known to those of skill in the art. Exemplary effector molecules include, but not limited to, a radiolabels, fluorescent markers, enzymatic markers or toxins (for example, Pseudomonas exotoxin (PE), see “Monoclonal Antibody-Toxin Conjugates: Aiming the Magic Bullet,” Thorpe et al., Monoclonal Antibodies in Clinical Medicine, Academic Press, pp. 168-190, 1982; Waldmann, Science, 252: 1657, 1991; U.S. Pat. No. 4,545,985 and U.S. Pat. No. 4,894,443, for a discussion of toxins and conjugation). Specific examples of detectable labels include a radioactive isotope, an enzyme substrate, a co-factor, a ligand, a chemiluminescent agent, a fluorescent agent, a hapten, or an enzyme.

Both covalent and noncovalent attachment means may be used to link an effector molecule to an antibody that binds a Spoc cell. The procedure for attaching an effector molecule to an antibody varies according to the chemical structure of the effector. Polypeptides typically contain a variety of functional groups; for example, carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of linker molecules such as those available from Pierce Chemical Company (Rockford, Ill.). The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (for example, through a disulfide linkage to cysteine) or to the alpha carbon amino and carboxyl groups of the terminal amino acids. Alternatively, the effector molecule can be linked to a secondary antibody that specifically binds the Spoc cell antibody.

In view of the large number of methods that have been reported for attaching a variety of radiodiagnostic compounds, radiotherapeutic compounds, label (for example, enzymes or fluorescent molecules), trophic factors, and other agents to antibodies, one skilled in the art will be able to determine a suitable method for attaching a given agent to an antibody. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P., Molecular Probes: Handbook of Fluorescent Probes and Research Chemicals (1992-1994). The metal compounds that can be conjugated to the antibodies include, but are not limited to, ferritin, colloidal gold, and particularly colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m (⁹⁹Tc), ¹²⁵I and amino acids comprising any radionucleotides, including but not limited to, ¹⁴C, ³H and ³⁵S.

### Method for Detecting Neuronal Precursor Cells or Sub-Populations of Spoc Cells

A method is provided for the in vitro or in vivo detection of neuronal precursor cells using specific binding agents, such as antibodies (for example monoclonal antibodies) that recognize cell surface markers. In one embodiment, the antibody used for detecting neuronal precursor cells, such as a sub-population of Spoc cells, is the 804 monoclonal antibody.

The in vitro detection method can screen any biological sample containing cells expressing the antigen recognized by the 804 monoclonal antibody. Such samples include, but are not limited to, tissue from biopsies, autopsies, and pathology specimens. Biological samples also include sections of tissues, such as frozen sections taken for histological purposes. Biological samples further include samples from the peripheral nervous system, the central nervous system, or skeletal muscle. Other biological samples that can be detected by the in vitro detection method include embryonic samples and samples of cultured cells that express the antigen bound by the 804 monoclonal antibody.

In one embodiment, the sample is contacted with a monoclonal antibody that binds a cell in the sample expressing an antigen bound by the 804 monoclonal antibody. The monoclonal antibody that specifically binds the antigen can be linked to a detectable label, thereby allowing the detection of a neuronal precursor cell. Alternatively, the sample is contacted with a secondary antibody that specifically binds the monoclonal antibody. The secondary antibody can be linked to a detectable label, thereby allowing the detection of a neuronal precursor cell.

An in vivo detection method can localize any cell in a subject that expresses the antigen recognized by the 804 monoclonal antibody. The cell can be in the peripheral nervous system, the central nervous system, or in skeletal muscle of a subject. In one embodiment, the 804 monoclonal antibody, or a humanized form of the 804 monoclonal antibody, is administered to the subject for a sufficient amount of time for the antibody to localize to the neuronal precursor cell in the subject and to form an immune complex with the antigen. In one embodiment, the immune complex is detected. In specific, non-limiting examples, detection of an immune complex is performed by radiolocalization, radioimaging, or fluorescence imaging. In another embodiment, the antibody is linked to an effector molecule. In one specific, non-limiting embodiment, the effector molecule is a detectable label. Specific, non-limiting examples of detectable labels include a radioactive isotope, an enzyme substrate, a co-factor, a ligand, a chemiluminescent agent, a fluorescent agent, a hapten, or an enzyme. Another specific example of a detectable label is technichium-99.

In one embodiment, the 804 monoclonal antibody and a secondary antibody are administered to the subject for a sufficient amount of time for the 804 monoclonal antibody to from an immune complex with the antigen on a neuronal precursor cell, and for the secondary antibody to form an immune complex with the 804 monoclonal antibody. In one embodiment, the 804 monoclonal antibody is complexed with the secondary antibody prior to their administration to the subject. In one specific, non-limiting embodiment, the secondary antibody is linked to a detectable label and the immune complex is detected.

The 804 monoclonal antibody can be labeled (for example with technichium-99) and used in technichium-99, PET (positron emission tomography), or SPECT (single-photon emission computerized tomography) scans to detect new neuronal growth following brain or spinal cord injury or disease. Thus, a labeled 804 monoclonal antibody is an important tool for the diagnosis and prognosis of subjects with a neurologic injury or disease.

### Method for Isolating Neuronal Precursor Cells or Sub-Populations of Spoc Cells

A method is also provided for isolating neuronal precursor cells, for example a sub-population of Spoc cells, wherein the cells are detected using specific binding agents, such as antibodies (for example, using the methods disclosed above) that recognize cell surface markers, and the neuronal precursor cells are isolated. This particular method of isolation of the neuronal precursor cells includes isolating cells from tissue of the peripheral nervous system, the central nervous system, or the skeletal muscle of a subject. In other embodiments, the cells can be isolated from embryonic tissue or complete embryos. Tissue can be prepared for the purpose of isolating or obtaining individual neuronal precursor cells, for example a sub-population of Spoc cells, by using methods well known to one of skill in the art. Examples of methods of tissue preparation include enzymatic digestion with enzymes such as collagenase, mechanical disruption using instruments such as hand-held or motor-driven homogenizers, or by chemical disruption using, for example, chelators of calcium and magnesium. Methods for isolating neuronal precursor cells, for example a sub-population of Spoc cells, using size exclusion and antibodies to c-kit, c-met, Sca1, CD34, and Pax(3/7) are described in U.S. patent application Ser. No. 10/003,400, filed Oct. 22, 2001, and published PCT Application No. 03/035838 A2.

As disclosed herein, neuronal precursor cells, for example a sub-population of Spoc cells, can be isolated using an antibody that specifically binds these cells. In one embodiment, neuronal precursor cells, or a sub-population thereof, are contacted with a monoclonal antibody that specifically binds a GRIN1 polypeptide. In other embodiments, neuronal precursor cells, or a sub-population thereof, are contacted with a monoclonal antibody that specifically binds a polypeptide having an amino acid sequence set forth as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In one specific non-limiting example, neuronal precursor cells, or a sub-population thereof, are contacted with the 804 monoclonal antibody and then the cells bound to the monoclonal antibody are isolated. In one embodiment, the use of an antibody that specifically binds a sub-population of Spoc cells can be combined with isolation of cells based on size.

In one embodiment, the sub-population of Spoc cells is isolated by passing digested skeletal muscle through a series of filters of varying pore size. The cells are passed through two filters, where a first filter has a pore size of about 50-200 μm, about 60-150 μm, about 80-100 μm, or about 100 μm and a second filter has a pore size of about 10-50 μm, 20-40 μm, or about 40 μm. In one embodiment, the isolated cells are less than 40 μm in diameter. In other embodiments, isolated cells are between about 3 μm and 10 μm in diameter. In another embodiment the isolated cells are about 4 μm in diameter.

The cells can be also sorted by size by passing them through size-exclusion columns. In one such embodiment, the cells are eluted along a size gradient such that the largest cells are eluted first and the smallest cells are eluted last. The cells can also be sorted by size using Fluorescence activated cell sorting (FACS). Cells of about 3 μm to 10 μm in diameter, or of about 4 μm in diameter, are isolated. The identity of these cells is then confirmed using an antibody that specifically binds a sub-population of Spoc cells, such as the 804 monoclonal antibody.

In another embodiment, neuronal precursor cells, for example a sub-population of Spoc cells, are isolated using an antibody disclosed herein. In one specific, non-limiting example, a sub-population of Spoc cells is isolated that includes cells that specifically bind to the 804 monoclonal antibody as greater than 20% of the population, greater than 30% of the population, greater than 40% of the population, greater than 50% of the population, greater than 80% of the population, greater than 90% of the population, greater than 95% of the population, or greater than 98% of the population of Spoc cells. In order to purify a sub-population of Spoc cells, the method can be performed a single time on a sample, or can be performed more than one time in succession.

In one embodiment, a suspension of cells, for example neuronal or muscle cells, is produced, and an antibody that specifically binds a neuronal precursor cell, such as the 804 monoclonal antibody, is reacted with the cells in suspension. For example, the antibodies can be conjugated to other compounds including, but not limited to, enzymes, magnetic beads, colloidal magnetic beads, haptens, fluorochromes, metal compounds, radioactive compounds or drugs.

FACS can be used to sort cells that express an antigen or cell surface marker of interest, by contacting the cells with an appropriately labeled antibody. Methods of determining the presence or absence of a cell surface marker are well known in the art. In one embodiment, additional antibodies and FACS sorting can further be used to produce substantially purified populations of neuronal precursor cells or sub-populations of Spoc cells. A FACS employs a plurality of color channels, low angle and obtuse light-scattering detection channels, and impedance channels, among other more sophisticated levels of detection, to separate or sort cells. Any FACS technique may be employed as long as it is not detrimental to the viability of the desired cells. (For exemplary methods of FACS see U.S. Pat. No. 5,061,620, herein incorporated by reference).

However, other techniques of differing efficacy may be employed to purify and isolate desired populations of cells. The separation techniques employed should maximize the retention of viability of the fraction of the cells to be collected. The particular technique employed will, of course, depend upon the efficiency of separation, cytotoxicity of the method, the ease and speed of separation, and what equipment and/or technical skill is required.

Separation procedures may include magnetic separation, using antibody-coated magnetic beads, affinity chromatography, cytotoxic agents, either joined to a monoclonal antibody or used in conjunction with complement, and “panning,” which utilizes a monoclonal antibody attached to a solid matrix, or another convenient technique. Antibodies attached to magnetic beads and other solid matrices, such as agarose beads, polystyrene beads, hollow fiber membranes and plastic petri dishes, allow for direct separation. Cells that are bound by the antibody can be removed from the cell suspension by simply physically separating the solid support from the cell suspension. The exact conditions and duration of incubation of the cells with the solid phase-linked antibodies will depend upon several factors specific to the system employed. The selection of appropriate conditions, however, is well within the skill in the art.

The unbound cells then can be eluted or washed away with physiologic buffer after sufficient time has been allowed for the cells expressing a marker of interest (for example, the antigen bound by monoclonal antibody 804) to bind to the solid-phase linked antibodies. The bound cells are then separated from the solid phase by any appropriate method, depending mainly upon the nature of the solid phase and the antibody employed.

Antibodies can be conjugated to biotin, which then can be removed with avidin or streptavidin bound to a support, or fluorochromes, which can be used with FACS, to enable cell separation (see above). In one example, the neuronal precursor cells initially can be separated from other cells by the cell-surface expression of the antigen bound by monoclonal antibody 804, for example a GRIN1 polypeptide. In one specific, non-limiting example, cells expressing a GRIN1 polypeptide are positively selected by magnetic bead separation, wherein magnetic beads are coated with monoclonal antibody 804. The neuronal precursor cells or the sub-population of Spoc cells are then removed from the magnetic beads.

Release of the neuronal precursor cells or the sub-population of Spoc cells from the magnetic beads can be affected by culture release or other methods. Purity of the isolated neuronal precursor cells or the sub-population of Spoc cells can be checked with a FACSCAN.RTM. flow cytometer (Becton Dickinson, San Jose, Calif.), for example, if so desired. In one embodiment, further purification steps are performed, such as FACS sorting the population of cells released from the magnetic beads on the basis of c-kit, c-met, Sca1, or CD34 expression. Panning can be used to separate cells that do not express c-kit, c-met, Sca1, or CD34, or react (or do not react) with an antibody, such as monoclonal antibody 804 (for panning methods see Small et al., J Immunol Methods 167(1-2): 103-107, 1994).

In one embodiment, the cells are selected by size (see above) and then the c-kit⁻c-met⁻CD34⁻Sca-1 Pax(3/7)⁻ subpopulation of Spoc cells are identified using the specific binding agents, such as antibodies that recognize the c-met, c-kit, CD34, or Sca1 cell surface markers.

In one embodiment the c-met, c-kit, CD34, and Sca1 antibodies are immobilized. A particular embodiment uses magnetic cell sorting. This method involves a combination of monoclonal antibodies which are covalently bound to the surface of magnetic beads and which are directed to cell surface markers which are absent from the cells being selected. For example, to isolate the c-kit^{-c-met}⁻CD34⁻Sca-1⁻Pax(3/7)⁻ Spoc cells or a sub-population of Spoc cells, monoclonal antibodies to c-met, c-kit, CD34, and Sca1 bound to magnetic beads are used. All cells expressing either c-met, c-kit, CD34, or Sca1, or any combination of these cell surface markers, will be bound by the antibodies and retained by the beads. Since the cells bound to the magnetic beads are immobilized by the magnet, the c-kit⁻c-met⁻CD34⁻Sca-1⁻ cells that remain in suspension can be isolated from the other cells. A monoclonal antibody that binds an antigen expressed on a sub-population of Spoc cells can then be used to isolate this sub-population of c-kit^{-c-met}⁻CD34⁻Sca-1⁻Pax(3/7)⁻ Spoc cells.

In another embodiment, purified populations of c-kit^{-c-met}⁻CD34⁻Sca-1⁻Pax(3/7)⁻ Spoc cells, or a sub-population of Spoc cells, are isolated via FACS. Fluorescent-tagged antibodies against c-met, c-kit, CD34, and Sca1 identify c-met⁺, c-kit⁺, CD34⁺, Sca1⁺, and cells expressing any combination of these cell surface markers (for example, c-met⁺c-kit⁺ double-positive populations of cells, c-met⁺c-kit⁺Sca1⁺ cells, or c-kit⁺CD34⁺Sca1⁺ cells) allowing for the identification and isolation of the c-kit^{-c-met}⁻CD34⁻Sca-1⁻ population. The cells can also be isolated on the basis of size. A sub-population of Spoc cells is then identified using an antibody that binds the sub-population of cells, such as monoclonal antibody 804.

In other embodiments antibodies can be covalently bound to inert beads, such as sepharose beads. The beads can be packed in a column or maintained as a slurry. The cells expressing one or more of the cell surface markers are recognized by the antibodies, become bound to the beads, and a sub-population of Spoc cells is identified.

In another embodiment the antibodies are not immobilized. In a particular embodiment the addition of the antibodies to a mixture of cells causes the aggregation of cells expressing the cell surface markers recognized by the antibodies. The cells not expressing the cell surface markers are excluded from the aggregates and can be isolated.

Neuronal precursor cells, or a sub-population of Spoc cells, isolated by these or other methods can be maintained in culture, and can be differentiated in culture. Methods for maintaining neuronal precursor cells, or a sub-population of Spoc cells, in culture and differentiating these cells are disclosed in U.S. patent application Ser. No. 10/003,400, filed Oct. 22, 2001, and published PCT Application No. 03/035838 A2, both of which are incorporated herein by reference in their entirety.

### Methods for Differentiating Neurons from Spoc Cells

A method is disclosed herein for differentiating Spoc cells, or a sub-population of Spoc cells, into neurons. In one embodiment, differentiation into neurons is induced by culturing Spoc cells that specifically bind to monoclonal antibody 804 in medium similar to a Spoc cell growth medium, but which does not include at least one growth factor, such as EGF or FGF. The Spoc cells can be from murine, porcine, or human skeletal muscle tissue.

In one embodiment, a sub-population of Spoc cells, that are isolated by being specifically bound by monoclonal antibody 804 are expanded on a solid substrate that permits the adhesion of a sub-population of cells in the presence of a culture medium. In one embodiment, the solid substrate is a container, such as a tissue culture dish. In another embodiment, the solid substrate is in the form of beads designed for tissue culture. The medium can be a growth medium, or any buffer that maintains the viability of the cells. A variety of culture media are known and are suitable for use. Generally, the growth medium includes a minimal essential medium. In one embodiment, the medium is DMEM or F12, or a combination of DMEM and F12 (at a ratio between about 1:1 to about 10:1 DMEM:F12).

The growth medium may be supplemented with serum. Specific, non-limiting examples of serum are horse, calf or fetal bovine serum. The medium can have between about 3% by volume to about 10% by volume serum, or about 5% by volume serum.

In one embodiment, the medium contains one or more additional additives such as nutrients. Specific, non-limiting examples of these nutrients are shown in the table below:

| Additive | Exemplary Concentration |
|---|---|
| Serum | About 3% to about 10% |
| Insulin | About 5 μg/ml to about 10 μg/ml |
| Transferring | About 5 μg/ml to about 10 μg/ml |
| Selenium | About 6 μg/ml |
| Ethanolamine | About 2 μg/ml |
| EGF | About 5 ng/ml to about 10 ng/ml |
| FGF | About 5 ng/ml to about 10 ng/ml |
| Gentamycin | About 25 μg/ml to about 50 μg/ml |
| fungizone | About 0.2 μg/ml to about 2.5 μg/ml |

The stem cell growth media can also be supplemented with growth factors. In one embodiment, the growth medium includes basic fibroblast growth factor (bFGF). In one specific example, the growth medium includes between about 2 ng/ml to about 100 ng/ml of bFGF, such as for example between about 5 ng/ml to about 50 ng/ml, between about 8 ng/ml to about 20 ng/ml, or between about 5 to about 10 ng/ml bFGF. In yet another example, the medium includes about 10 ng/ml bFGF. In another embodiment, the growth medium includes epidermal growth factor (EGF). In one specific example, the growth medium includes between about 2 ng/ml to about 100 ng/ml of EGF, such as for example between about 5 ng/ml to about 50 ng/ml, between about 8 ng/ml to about 20 ng/ml, or between about 5 ng/ml to about 10 ng/ml EGF. In yet another example, the medium includes about 10 ng/ml EGF. Thus in one embodiment, the growth medium is 1:1 DMEM/F12 and includes 5% fetal bovine serum, 10 ng/ml FGF, 10 ng/ml EGF, 5 μg/ml insulin, 5 μg/ml transferrin, 6 ng/ml selenium, 2 μg/ml ethanolamine.

In one specific, non-limiting example, the cells are cultured in the growth medium for about 1 day to about 5 days. In other specific, non-limiting examples, the cells are cultured in the growth medium for about 2 days to about 4 days, or for about 3 days.

Following growth of the sub-population Spoc cells, the cells expressing the antigen recognized by the 804 monoclonal antibody are specifically bound by the 804 monoclonal antibody and are isolated from the Spoc cells that do not express the antigen (see above), yielding two sub-populations of Spoc cells, those that bind the 804 monoclonal antibody and that can differentiate into neuronal cells (the neuronal precursor sub-population), and those that do not bind the 804 monoclonal antibody and that can differentiate into cardiomyocytes (the cardiomyocyte precursor sub-population). The isolated sub-population of neuronal precursor cells is then differentiated into neuronal cells using a differentiation medium or through maintenance of the cells in the original medium containing EGF and/or bFGF. A differentiation medium is a growth medium that lacks at least one growth factor. Growth factors removed from the medium include, but are not limited to, bFGF or EGF, or a combination of bFGF and EGF.

Removal of at least one growth factor causes the cells to adhere to the tissue culture dish and acquire characteristics of a differentiated neuron (for example, expression of beta-3-tubulin). However, the maintenance of the 804 positive sub-population of Spoc cells in the original culture media (containing EGF and bFGF) will also lead to neuronal cell differentiation, but with different kinetics. Differentiation refers to the process whereby relatively unspecialized cells, such as the c-met⁻c-kit⁻CD34⁻Sca1⁻Pax(3/7)⁻ muscle-derived stem cells, acquire specialized structural and/or functional features characteristic of mature cells, such as neurons.

Differentiation of c-met⁻c-kit⁻CD34⁻Sca1⁻Pax(3/7)⁻ Spoc cells, or a sub-population of Spoc cells, into neurons can be measured by any method known to one of skill in the art. Specific, non-limiting examples are immunohistochemical analysis to detect expression of neuronal polypeptides (for example, β-tubulin, neurofilament, neuron specific enolase, serotonin, dopamine, tyrosine hydrolase, or other neuron specific markers), or assays such as ELISA assay and Western Blot analysis. Differentiation of cells can also be measured by assaying the level of mRNA coding for neuronal polypeptides using techniques such as Northern Blot, RNase protection, and RT-PCR. In another embodiment, the electrophysiological properties of the cells (synaptic transmission, voltage potentials, or action potentials) are assessed.

### Methods for Screening for Agents that Affect Neuronal Cell Function or Differentiation

Also disclosed are methods for screening for agents that affect neuronal cell function or differentiation. The method includes contacting an isolated sub-population of Spoc cells or a neuronal cell precursor with the compound or agent, and assaying the neuronal cell function or differentiation. A change in the growth, differentiation, or activity of the isolated sub-population of Spoc cells or a neuronal cell precursor indicates that the compound is a modulator of neuronal cell function or differentiation.

The compounds which may be screened in accordance with this disclosure include, but are not limited to peptides, antibodies and fragments thereof, and other organic compounds (for example, peptidomimetics, small molecules) affect neuronal cell function or differentiation. Such compounds may include, but are not limited to, peptides such as, for example, soluble peptides, including but not limited to members of random peptide libraries; (see, for example, Lam et al., Nature, 354:82-84, 1991; Houghten et al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries; see, for example, Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small organic or inorganic molecules.

Other compounds which can be screened include but are not limited to small organic molecules that are able to gain entry into a sub-population of Spoc cells or neuronal precursor cells, and affect neuronal cell differentiation; or such compounds that affect an activity or function of a sub-population of Spoc cells or neuronal precursor cells.

Computer modeling and searching technologies permit identification of compounds, or the improvement of already identified compounds that can affect neuronal cell function or differentiation. Examples of molecular modeling systems are the CHARMM and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

### Methods for Treatment of Neurologic Diseases and/or Neurodegenerative Disorders

In other embodiments, methods are provided for treating a subject suffering from a disease or a disorder, such as a neurologic injury or neurodegenerative disorder, or alleviating the symptoms of such a disorder, by administering cells isolated and/or cultured according to the methods disclosed. The cells can be a sub-population of Spoc cells (c-kit^{-c-met}⁻CD34⁻Sca1⁻Pax(3/7)⁻804⁺ cells), neuronal precursor cells, or differentiated neuronal cells. The cells can be autologous or heterologous.

The present methods can be employed to isolate neuronal precursor cells and to produce neuronal cells in order to deliver the cells, or molecules expressed by these cells, to the brain for diagnosis, treatment or prevention of disorders or diseases of the CNS, brain, and/or spinal cord. These disorders can be neurologic or psychiatric disorders. These disorders or diseases include brain diseases such as Alzheimer's disease, Parkinson's disease, Lewy body dementia, multiple sclerosis, epilepsy, cerebellar ataxia, progressive supranuclear palsy, amyotrophic lateral sclerosis, affective disorders, anxiety disorders, obsessive compulsive disorders, personality disorders, attention deficit disorder, attention deficit hyperactivity disorder, Tourette Syndrome, Tay Sachs, Nieman Pick, and other lipid storage and genetic brain diseases and/or schizophrenia. The method can also be employed in subjects suffering from or at risk for nerve damage from cerebrovascular disorders such as stroke in the brain or spinal cord, from CNS infections including meningitis and HIV, from tumors of the brain and spinal cord, or from a prior disease. The method can also be employed to deliver agents to counter CNS disorders resulting from ordinary aging (for example, insomnia or loss of the general chemical sense), brain injury (including traumatic brain injury (TBI)), or spinal cord injury. The present method can be employed to deliver cells to the brain for diagnosis, treatment or prevention of neurodegenerative disorders.

The application of cells can also be used to replace or augment peripheral cells and neurons that are injured by neurotoxins and other insults. This method of treatment is beneficial in cases of Alzheimer's disease where an environmental factor is suspected of being one of the causative agents of the disease. Application of cells can also be used to affect the loss of smell or of the general chemical sense which may be associated with neurodegenerative diseases and ordinary aging.

The cells can also be used in the treatment of Parkinson's disease. The principal therapeutic target in the brain for Parkinson's disease is the substantia nigra which extends forward over the dorsal surface of the basis peduncle from the rostral border of the pons toward the subthalamic nucleus. Other therapeutic target areas are the locus ceruleus which is located in the rostral pons region and the ventral tegmental area which is located dorsomedial to the substantia nigra.

After the cells, for example differentiated neuronal cells, are isolated according to the isolation and/or cell culturing method described above, the cells are suspended in a physiologically compatible carrier. The carrier can be any carrier compatible with the other ingredients of the formulation and not deleterious to the recipient thereof. Those of skill in the art are familiar with physiologically compatible carriers. Examples of suitable carriers include cell culture medium (for example, Eagle's minimal essential media), phosphate buffered saline, and Hank's balanced salt solution +/−glucose (HBSS).

The volume of cell suspension administered to a subject will vary depending on the site of implantation, treatment goal and amount of cells in solution. Typically the amount of cells administered to a patient will be a therapeutically effective amount. For example, where the treatment is for Parkinson's disease, transplantation of a therapeutically effective amount of cells will typically produce a reduction in the amount and/or severity of the symptoms associated with that disorder, for example, rigidity, akinesia and gait disorder.

In one example, a severe Parkinson's patient needs at least about 100,000 surviving dopamine cells per grafted site to have a substantial beneficial effect from the transplantation. As cell survival is low in brain tissue transplantation in general, (5-10%) at least 1 million cells are administered, such as from about 1 million to about 4 million dopaminergic neurons are transplanted.

In one embodiment, the cells are administered to the subject's brain. The cells may be implanted within the parenchyma of the brain, in the space containing cerebrospinal fluids, such as the sub-arachnoid space or ventricles, or extaneurally. Thus, in one example, the cells are transplanted to regions of the subject which are not within the central nervous system or peripheral nervous system, such as the celiac ganglion or sciatic nerve. In another embodiment, the cells are transplanted into the central nervous system, which includes all structures within the dura mater.

Typically, the cells are administered by injection into the brain of the subject. Injections can generally be made with a sterilized syringe having an 18-21 gauge needle. Although the exact size needle will depend on the species being treated, the needle should not be bigger than 1 mm diameter in any species. Those of skill in the art are familiar with techniques for administering cells to the brain of a subject.

In other embodiments, methods are provided for treating a subject suffering from a disease or a disorder, such as a neurologic injury or neurodegenerative disorder, or alleviating the symptoms of such a disorder, by administering the 804 monoclonal antibody. For example, the 804 monoclonal antibody can be used to deliver trophic factors to de-repress nascent neuronal growth in a variety of conditions, for example stroke, Parkinson's disease, and spinal cord injury.

The disclosure is illustrated by the following non-limiting examples.

### EXAMPLES

### Example 1

### Method of Isolating and Expanding Cardiomyocyte Precursor Cells from Adult Mouse Skeletal Muscle and Their Use to Produce Antibodies

Skeletal muscle tissue from hind legs of 6-10 week-old male C57B1/SJ6 mice was cut into small pieces and digested with collagenase for two hours at 37° C. The digested tissue was cleared of cell debris and other undigested tissue fragments by passage through a 100 μm filter and then through a 40 μm filter (Falcon). The cell suspension was centrifuged at low speed (1,400 rpm) to clear as much of the small muscle fiber fragments as possible. The cells at this stage consisted mostly of clusters of small round cells approximately 4 μm in diameter, called Spoc (skeletal-based precursors of cardiomyocytes) cells.

The Spoc cells were plated at a density of approximately 10⁵ cells per cm² in regular tissue culture dishes in complete growth medium (1:1 DMEM/F12 supplemented with 5% fetal bovine serum (FBS), 10 ng/ml human EGF, 10 ng/ml human bFGF (PeproTech, Inc.), 5 μg/ml insulin, 5 μg/ml transferrin, 6 ng/ml selenium, 2 μg/ml ethanolamine (ITS-X, Invitrogen Corporation), 25 μg/ml gentamicin and 2.5 μg/ml fungizone (Life Technologies)). After a few days, the culture consisted of a floating population of round cells and some adherent fibroblasts. The round cells enlarged as they underwent a few rounds of cell division during which time they became clusters of floating round cells with an increased diameter of 10-14 μm. The cells in these clusters were referred to as CS (cardiac precursors from Spoc) cells (see PCT Patent Application No. PCT/USO2/33860, filed Oct. 22, 2002, herein incorporated by reference).

These cells were then used to produce rat monoclonal antibodies using standard procedures. Briefly, mice were injected subcutaneously five times at two to three week intervals with 1-10×10⁶ Spoc cells in phosphate buffered saline, pH 7.4. One rat was injected subcutaneously and intraperitoneally with 5×10⁶ cells three days prior to the fusion.

Splenocytes were fused with P3×63-Ag8.653 myeloma cells using 50% polyethylene glycol. Cells were cultured and a hybridoma library of HAT-selected cells was isolated essentially as described in Kenney, et al. The hybridoma library was cloned using a fluorescent activated cell sorter with automatic cell deposition unit. Single viable cells were sorted into 96-well plates based upon the analysis criteria of forward-scatter, side-scatter and propidium iodide fluorescence (see Kenney et al., Biotechnology 13:787-90, 1995).

Approximately 1500 clones from the two libraries were expanded to the extent that supernatant from each could be used against fixed Spoc cells in a fluorescent assay. Pools of four supernatants were used against each slide of Spoc cells that had been deposited on silanated slides in a cytospin machine and fixed with 4% paraformaldehyde. Detection was accomplished with a FITC-labeled secondary goat anti-rat IgG and IgM antibody. Pooled samples that suggested positive reaction were broken down into their component supernatants which were tested individually in the same manner. The 804 clone was positive for a subset of Spoc cells after multiple experiments and was then grown in an expanded culture. The reacting dominant antibody was purified and identified as an IgM antibody.

Thus, one particular clone, known as 804, produced an IgM monoclonal antibody that reacted strongly against a cell surface antigen on a subset of Spoc cells (This antibody was deposited with American Type Culture Collection in accordance with the Budapest Treaty on Mar. 24, 2004, ATCC Deposit No. PTA-5888). This antibody, together with the Sca 1 monoclonal antibody was then used to isolate all four possible combinations of Sca 1/804 antigenicity, for instance, ++, +−, −−, −+.

When the Sca 1⁻/804⁻purified cells were grown in isolation, they developed into beating cells in culture, while the Sca1⁻/804⁺ cells in culture grew into neuronal cells expressing neuronal markers such as Beta-3 tubulin.

### Example 2

### Detection of Spoc cells Using Immunohistochemistry

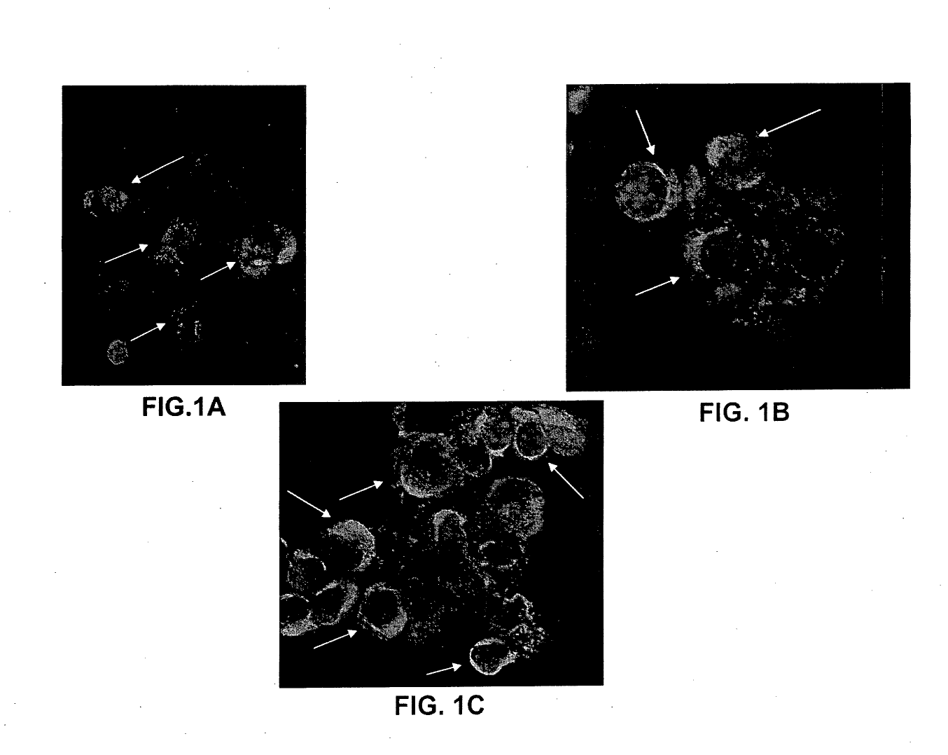

Spoc cells were plated at a density of approximately 10⁵ cells per cm² in regular tissue culture dishes in complete growth medium (1:1 DMEM/F12 supplemented with 5% fetal bovine serum (FBS), 10 ng/ml human EGF, 10 ng/ml human bFGF (PeproTech, Inc.), 5 μg/ml insulin, 5 μg/ml transferrin, 6 ng/ml selenium, 2 μg/ml ethanolamine (ITS-X, Invitrogen Corporation), 25 μg/ml gentamicin and 2.5 μg/ml fungizone (Life Technologies)). After a few days, the culture consisted of a floating population of round cells and some adherent fibroblasts. The round cells enlarged as they underwent a few rounds of cell division during which time they became clusters of floating round cells with an increased diameter of 10-14 μm. The cells in these clusters are CS (cardiac precursors from Spoc) cells. Monoclonal antibody 804 specifically bound a sub-population of Spoc cells cultured for 0-4 days (FIGS. 1A, 1B, and 1C). In addition, monoclonal antibody 804 bound a few cells after 5 days of culture. However, by this time (5 days) the vast majority of cells are 804 negative, unless the 804 positive cells have been isolated previously from the 804 negative cells (those cells which in turn will develop into beating cardiomyocytes). Thus, the cardiomyocyte precursors will suppress the 804 positive Spoc cells from differentiating into neuronal cells when the two precursor subsets (804 positive and negative) are co-cultured.

Specimens of Spoc cells or tissue sections were air-dried for 30 minutes and then fixed in 4% paraformaldehyde at 4° C. followed by a rinse for 5 minutes with phosphate buffered saline. (PBS). Alternatively, for mice, paraformaldehyde infusion was performed. Sections were blocked with goat serum for 30 minutes and then incubated overnight, at 4° C., with either a monoclonal raised against Spoc cells, anti-b-tubulin, or another primary antibody. Following the overnight incubation, the specimens were rinsed 3 times (5 minutes each) with PBS and blocked again with goat serum for 30 minutes. The specimens were then incubated at room temperature with a secondary antibody, conjugated with either horseradish peroxidase, alkaline phosphatase, Fluorescein Isothiocyanate (FITC), Texas Red, or Tetramethylrhodamine Isothiocyanate (TRITC), for 1 hour. They were again rinsed 3 times (5 minutes each) with PBS and then visualized with a laser confocal microscope (Leica) to detect fluorescent signals.

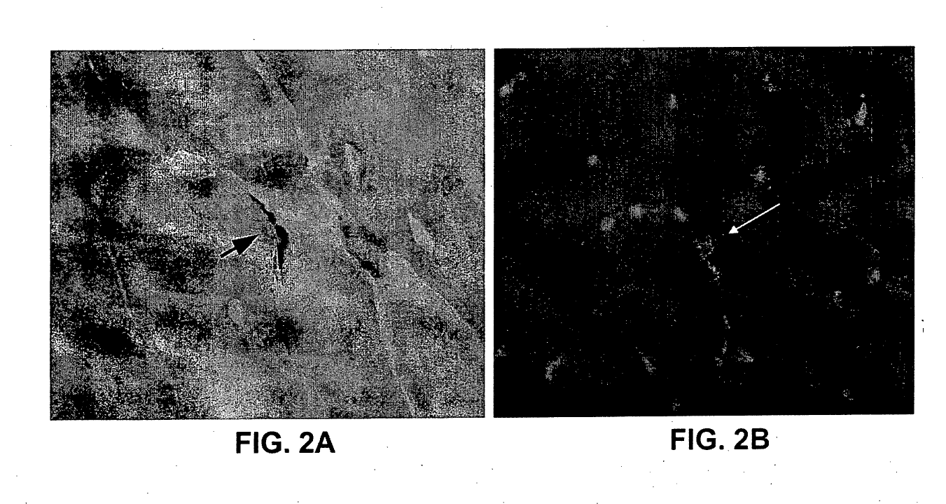

The 804 marker was used in fluorescent and histochemical studies to identify the cells in mouse skeletal muscle (FIG. 2A). These same cells were then viewed under the electron microscope (EM) using immuno-gold to identify the antigen against which 804 reacts. This study showed that 804 antigen is located in part in caveolae on the cell surface. Double labeling with a caveolin immuno-gold antibody showed the co-localization. This suggested the possibility that the antigen, which had been determined to be in the triton insoluble membrane fraction, was a GPI anchored protein. Indeed it was found that the 804 antigen could be cleaved from the cell surface using phospholipase-C, a mark of a GPI linked protein.

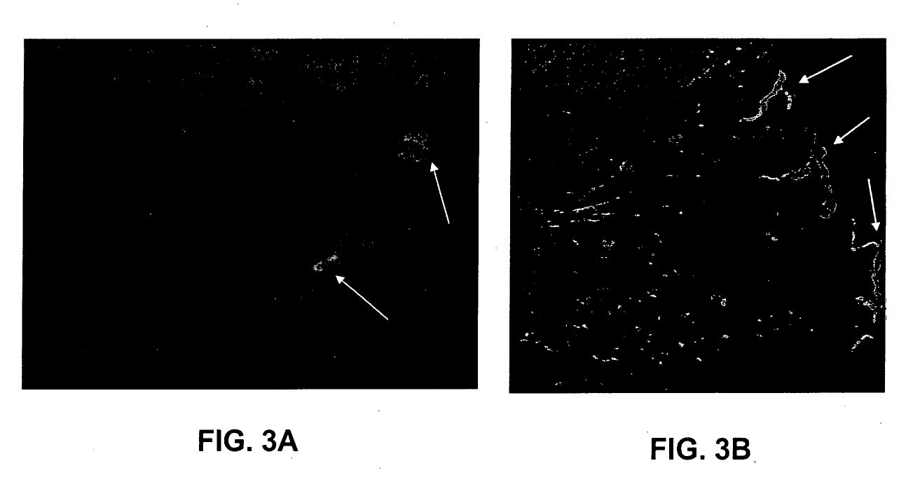

The 804 antigen has been shown to identify the homologous cells in human skeletal muscle and pig (for example, see FIG. 2B). Moreover, 804+ cells isolated from pig skeletal muscle and cultured for 12 days formed a neurosphere and expressed neuronal-specific markers (FIG. 3A). The antibody also identifies a small number of 804+ cells in mouse and pig hippocampus. More copious however, in pig, mouse, and human hippocampus, are more mature cells with an astrocyte like morphology that react with the 804 antibody. These cells could be the neuronal stem cells that have been described as being present in restricted numbers in three parts of the brain: the CA1 region of the hippocampus, the olfactory lobe, and the subventricular region of the lateral ventricle. In support of this assertion, the 804 antibody detects this sub-population in these three regions of the mouse brain and is not present elsewhere (for example, see FIG. 3B). Thus it is likely that in the brain, a small number of 6 μm, 804 positive cells divide asymmetrically to produce the neuronal precursors with an astrocyte morphology.

### Example 3

### Identification and Further Characterization of the Antigen Recognized by the 804 Monoclonal Antibody

As described above, the 804 monoclonal IgM antibody identified a subset of cells from skeletal muscle that, when separated from a Sca1 negative fraction of total Spoc cells, and subsequently cultured with FGF and EGF, develop into neuroepithelial cells in culture. The 804 antigen is continuously expressed on the soma and neuritic extensions of these cells as they progress from undifferentiated stem cells to differentiated neuronal cells.

In order to identify and further characterize the 804 antigen, the antibody was used to stain serial longitudinal sections of mouse brain, as well as pig and human hippocampus. Cells were fixed in 4% paraformaldehyde at 4° C. or in acetone at −20° C. for 10 minutes, then washed in PBS for a total of 15 minutes (3 times). Blocking was performed for 30 minutes at room temperature with either 3% BSA in PBS, 5% goat serum in PBS, or 10% goat serum in PBS. Incubation with primary antibody was performed at 4° C. overnight. Cells were then washed for a total of 15 minutes (3 times), in 1×PBS. Incubation with secondary antibody (with either FITC or Texas Red conjugation) was done at room temperature for 1 hour. Afterwards, cells were washed 3 times with PBS. Fluorescent mounting medium with DAPI (Vector) and cover slip were then placed over the sample. Images of cells were obtained with a Zeiss 200M Axiovert Fluorescent microscope.

The 804 antibody stained a subset of neurons in the olfactory lobe, the Dentate Gyrus of the hippocampus, and the sub-ependymal layer of the lateral ventricle in the mouse brain. These are the 3 regions of the brain known to contain neuronal stem cells (Schinder and Gage, Physiology 19: 253-261, 2004). The 804 antibody was also shown to identify a similar subset of cells in human and pig hippocampus, confirming the evolutionary conservation of this precursor cell marker.

The 804 antibody was also used to stain a series of mouse embryos from day ten onward using the 804 antibody and MACS (Magnetic Activated Cell Sorting). Dissociated embryonic cells were incubated (with gentle rocking) with the 804 antibody (1:50 concentration) in HBSS for 20 minutes at 4° C. Cells were then washed with bioin-free DPBS/BSA and spun down in a centrifuge. Cells were then incubated with biotinylated anti-rat IgG/IgM (1:50) for 20 minutes. DPBS/BSA was used to wash the cells, which were then spun down. Streptavidin Microbeads (Miltenyi) were then added to the cells for 20 minutes. The cell/bead suspension was then placed on Miltenyi Magnetic columns in the presence of a Milteny magnet. The flow-through (negative cells) was collected, and the column washed with DPBS/BSA. The column was removed from the magnet, and the 804 positive cells were forced free of the column by using a plunger.

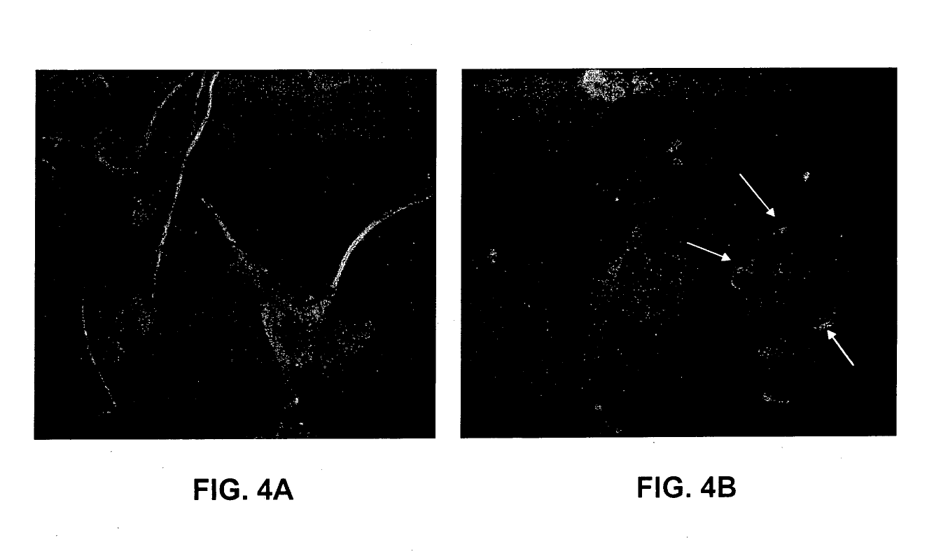

By day 12.5 of embryonic development, the 804 antigen is positive (804⁺) in limited amounts within the brain, developing spinal cord, heart, and base of the developing tongue. The use of the 804 antibody to isolate cells from dissociated total cells of the 12.5 day mouse embryos yields small undifferentiated cells that are indistinguishable from Spoc cells. Under culture conditions identical to those used for Spoc cells, the isolated 804⁺ embryonic cells develop neuronal morphologies and stain positive for the 804 antigen throughout their differentiation (FIG. 4A). Within days of culture, before the development of neuronal morphology, these cells began to express Beta-3 tubulin (a specific marker of neuronal cells).

In order to identify the antigen to which the 804 antibody reacts, immunoprecipitation from mouse hippocampus homogenate was performed. Immunoprecipitation yielded a clean band with an electrophoretic mobility of about 115 kDa. This band was excised and, after tryptic digest, subjected to mass spectroscopy. A set of peptides from a variety of cytoskeletal proteins including actin and all four alpha actinins were found to be present. In addition, a single additional transmembrane protein with a cell surface recognition site, a requisite for the target of the 804 antibody, was shown to be present. The peptides identified by mass spectroscopy include: KYADGVTGRV, KVLQFDPGTKN, KHNYESAAEAIQAVRD, and KIVNIGAVLSTRK. These peptides were a perfect match for the sequence of a subunit of the mouse NMDA receptor, which is referred to as GRIN1.

In order to confirm the identification of the GRIN 1 antigen, a commercial antibody to GRIN 1 (Anti-NMDAR (NR1 subunit) clone R1JHL from Phosphosolutions, Aurora, Colo.) was used to immunoprecipitate protein from hippocampus and Spoc cells and both products probed with the 804 antibody. Spoc cells were grown on tissue culture dishes in DMEM-F12 supplemented with EGF and FGF. Cells were collected and pelleted, then lysed with NP-40 Cell Lysis Buffer (50 mM Tris-HCl pH 8.0, 150 mM NaCl, and 1% NP-40 containing one Complete Protease Inhibitor pill per 10 ml) at 4° C. Hippocampus lysate and Spoc cell lysate were microcentrifuged at maximum speed for 15 minutes and supernatant removed. Antibody 804 or NR1 antibody was added (30 μg per ml of lysate) and mixed with lysate overnight at 4° C. Protein A/G (Sigma) was added (100 μl per 1 ml lysate) and mixed for 2 hours at 4° C. The Protein A/G with the bound antibody was recovered in a Pierce spin column and then washed 3 times with PBS at 4° C. The Protein A/G was then brought up in 1:1 Lamelli buffer and PBS, and boiled at 100° C. for two minutes. The supernatant was collected and run on Tris-Hepes-SDS gels (Gradipore) and immunoprecipitated protein was either visualized directly on the gel using colloidal Coomassie stain (Gradipore), or transferred to a membrane using a Novex apparatus. The Western blot was probed with the appropriate antibody and visualized using ECL reagent (Amersham).

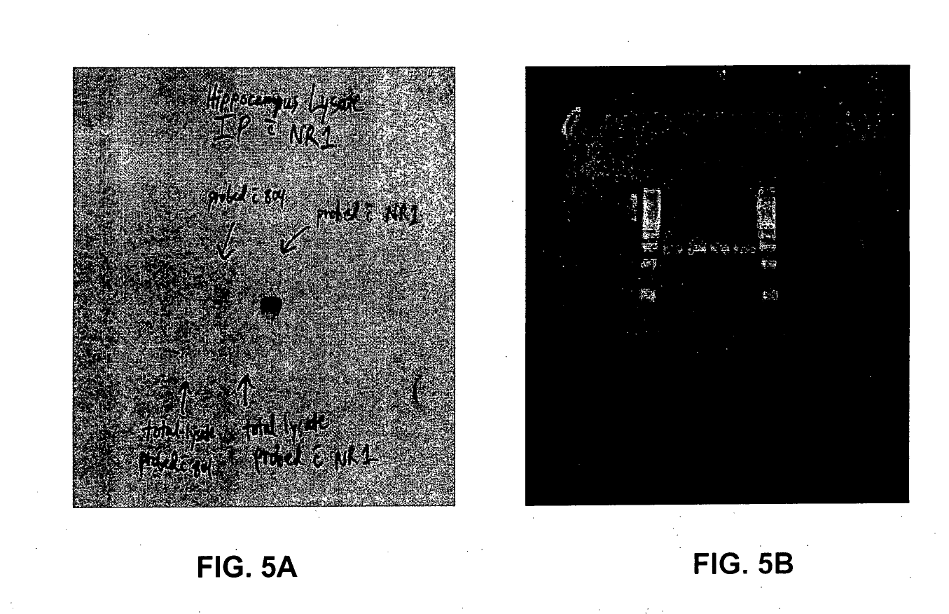

FIG. 5A shows hippocampus lysate immunoprecipitated with the GRIN1 antibody and probed with both the 804 and GRIN1 antibody. Both antibodies detect the same 115 kDa band. The control lanes showing no reaction to total lysate confirms the specificity of the immunoprecipitation using the GRIN1 antibody.

It is clear however, that because of the ubiquitous nature of GRIN1 and the rare and specific nature of the 804 antibody, that the antigen bound by the latter must be an isoform of GRIN 1. To confirm this, whole mouse brains were stained with GRIN1 antibody, which as expected, stains the brain uniformly. A second staining with 804 shows only a small subset of these cells in the aforementioned locations co-staining with 804. The size of the 804/GRIN1 antigen is within 10 kDa of the predicted size allowing for a post-translational modification such as glycosylation. This would be consistent with the fact that the 804 antibody is an IgM immunoglobulin, the class of which often binds to a glycosylated epitope. Co-staining of 804+Spoc cells isolated from mouse skeletal muscle, with the 804 monoclonal antibody and the GRIN1 antibody (FIG. 4B) provides further evidence that the antigen recognized by the 804 monoclonal antibody is an isoform of GRIN1.

In summary, the 804 antibody has been shown to bind to an isoform of the GRIN 1 subunit of the NMDA neuronal receptor present on undifferentiated pre-neuronal stem cells present in the developing mouse embryo, adult hippocampus of mice, pigs and human, and skeletal muscle of mouse pigs and human. This antibody identifies a stem cell that can be used to grow neuronal cells from these species.

It will be apparent that the precise details of the methods or compositions described may be varied or modified without departing from the spirit of the described invention. We claim all such modifications and variations that fall within the scope and spirit of the claims below.

1. A method of detecting the presence of a neuronal precursor cell, comprising
contacting a sample with a monoclonal antibody that binds a cell in the sample expressing an antigen bound by monoclonal antibody 804; and
detecting the cell in the sample bound by the monoclonal antibody, thereby detecting the neuronal precursor cell.

2. The method of claim 1, wherein the monoclonal antibody is produced by a hybridoma cell line originally deposited as ATCC Deposit No. PTA-5888.

3. The method of claim 1, wherein the monoclonal antibody specifically binds at least eight consecutive amino acids of a polypeptide at least 95% identical to SEQ ID NO: 1.

4. The method of claim 1, wherein the monoclonal antibody is labeled.

5. The method of claim 1, further comprising contacting the sample with a second antibody that specifically binds the monoclonal antibody.

6. The method of claim 5, wherein the second antibody is labeled.

7. The method of claim 1, wherein the monoclonal antibody binds at least eight consecutive amino acids of SEQ ID NO: 1.

8. The method of claim 1, wherein the sample comprises cells of the peripheral nervous system, cells of the central nervous system, or skeletal muscle cells.

9. The method of claim 8, wherein the sample comprises isolated c-kit⁻c-met⁻CD34⁻Sca1⁻Pax(3/7)⁻ neuronal precursor cells of skeletal muscle origin.

10. The method of claim 9, wherein the neuronal precursor cell is between about 3 μm and 10 μm in diameter.

11. The method of claim 1, wherein the neuronal precursor cell is a human, mouse, or porcine cell.

12. The method of claim 1, wherein the neuronal precursor cell is a human cell.

13. The method of claim 1, further comprising isolating the cell bound by the monoclonal antibody.

14. The method of claim 1, wherein the sample comprises skeletal muscle cells.

15. The method of claim 1, wherein the monoclonal antibody binds a GRIN1 polypeptide, but binds only a subset of cells expressing SEQ ID NO: 1.

16. The method of claim 1, wherein the monoclonal antibody binds at most 30% of cells expressing SEQ ID NO: 1.

17. The method of claim 1, wherein the monoclonal antibody binds a glycosylated form of SEQ ID NO: 1.

18. A method for isolating a neuronal precursor cell, comprising
(a) contacting a sample with a monoclonal antibody that specifically binds the antigen bound by a monoclonal antibody produced by a hybridoma from a cell line originally deposited as ATCC Deposit No. PTA-5888, under conditions wherein the monoclonal antibody specifically binds cells in the sample; and
(b) isolating cells bound to the monoclonal antibody,
thereby isolating neuronal precursor cells.

19. The method of claim 18, wherein step (a) comprises contacting the sample with the monoclonal antibody produced by a hybridoma from a cell line originally deposited as ATCC Deposit No. PTA-5888.

20. The method of claim 18, wherein isolating cells bound to the monoclonal antibody comprises using fluorescence activated cell sorting.

21. The method of claim 18, wherein isolating cells bound to the monoclonal antibody comprises contacting cells bound to the antibody with a solid substrate that binds the antibody.

22. The method of claim 18, wherein the antigen is a polypeptide comprising at least eight consecutive amino acids of a polypeptide at least 95% identical to SEQ ID NO: 1.

23. The method of claim 18, wherein the antigen is a polypeptide comprising at least eight amino acids of SEQ ID NO: 1.

24. The method of claim 18, wherein the antigen is a glycoprotein.

25. A method for producing a neuronal cell in vitro, comprising
isolating cells from a population of c-kit^{-c-met}⁻CD34⁻Sca1⁻Pax(3/7)⁻ cells from skeletal muscle, wherein the isolated cells are bound in vitro by an antibody that specifically binds the antigen bound by a monoclonal antibody produced by a hybridoma from a cell line originally deposited as ATCC Deposit No. PTA-5888,
culturing the cells bound by the antibody,
thereby producing a neuronal cell in vitro.

26. The method of claim 25, wherein the antibody is produced by a hybidoma originally deposited as ATCC Deposit No. PTA-5888.

27. The method of claim 25, wherein the c-kit^{-c-met}⁻CD34⁻Sca1⁻Pax(3/7)⁻ cells are human cells.

28. The method of claim 25, wherein the neuronal cell produces β-tubulin.

29. A method of generating neuronal cells in vitro, comprising
contacting a suspension of cells from an embryo with an antibody that specifically binds the antigen bound by monoclonal antibody produced by a hybridoma from a cell line originally deposited as ATCC Deposit No. PTA-5888 in vitro;
identifying precursor cells specifically bound by the antibody;
culturing the precursor cells in vitro, thereby generating neuronal cells.

30. The method of claim 29, wherein the antibody is produced by a hybridoma from a cell line originally deposited as ATCC Deposit No. PTA-5888.

31. The method of claim 29, wherein the neuronal cells express a polypeptide comprising an amino acid sequence set forth as SEQ ID NO: 1.

32. The method of claim 29, wherein the antigen is glycosylated.

33. The method of claim 29, wherein the antigen is a polypeptide at least 95% identical to SEQ ID NO: 1.

34. A method for treating a subject with a neurologic disorder, comprising,
providing a therapeutically effective amount of c-kit^{-c-met}⁻CD34⁻Sca1⁻Pax(3/7)⁻804⁺ cells to the subject, thereby alleviating a symptom of the neurodegenerative disorder.

35. The method of claim 34, wherein the neurologic disorder is a neurodegenerative disorder.
